SEQUENCE LISTING (18 sequences)

SEQ ID NO: 1 - DNA, 19 bp, synthetic construct
  source 1..19: mol_type = other DNA
gatcttcagccccgatgga

SEQ ID NO: 2 - DNA, 20 bp, synthetic construct
  source 1..20: mol_type = other DNA
caagggttgctctggcttgt

SEQ ID NO: 3 - DNA, 19 bp, synthetic construct
  source 1..19: mol_type = other DNA
tggcgagccttagtttgga

SEQ ID NO: 4 - DNA, 20 bp, synthetic construct
  source 1..20: mol_type = other DNA
tcgacatggagctggtgaaa

SEQ ID NO: 5 - DNA, 23 bp, synthetic construct
  source 1..23: mol_type = other DNA
cctgacgctgaagtatccgatag

SEQ ID NO: 6 - DNA, 21 bp, synthetic construct
  source 1..21: mol_type = other DNA
ttttccatgtcgtcccagttg

SEQ ID NO: 7 - DNA, 20 bp, synthetic construct
  source 1..20: mol_type = other DNA
tagagtgcaaggagttcggg

SEQ ID NO: 8 - DNA, 20 bp, synthetic construct
  source 1..20: mol_type = other DNA
ccggcatatacgagtgtgaa

SEQ ID NO: 9 - DNA, 20 bp, synthetic construct
  source 1..20: mol_type = other DNA
tggatacctgagcatcacca

SEQ ID NO: 10 - DNA, 19 bp, synthetic construct
  source 1..19: mol_type = other DNA
ctgctacctgcactttgcc

SEQ ID NO: 11 - DNA, 20 bp, synthetic construct
  source 1..20: mol_type = other DNA
aaaccagagattcccacacg

SEQ ID NO: 12 - DNA, 20 bp, synthetic construct
  source 1..20: mol_type = other DNA
gtcgtgggtcttcctgtagc

SEQ ID NO: 13 - DNA, 19 bp, synthetic construct
  source 1..19: mol_type = other DNA
gggtgggaaccgtgtcatt

SEQ ID NO: 14 - DNA, 17 bp, synthetic construct
  source 1..17: mol_type = other DNA
cttgcggcttggcttca

SEQ ID NO: 15 - DNA, 20 bp, synthetic construct
  source 1..20: mol_type = other DNA
gcttcaggagctgaataccc

SEQ ID NO: 16 - DNA, 20 bp, synthetic construct
  source 1..20: mol_type = other DNA
aaggatcacgaccatcttcc

SEQ ID NO: 17 - DNA, 20 bp, synthetic construct
  source 1..20: mol_type = other DNA
ccacagtcacctatggcaac

SEQ ID NO: 18 - DNA, 20 bp, synthetic construct
  source 1..20: mol_type = other DNA
agtgtctcctggctctggtt

(19) United States
(12) Patent Application Publication
(10) Pub. No.: US 2024/0245661 A1
(43) Pub. Date: Jul. 25, 2024

(54) MCT4-TARGETED THERAPEUTICS PREVENTING AKI TO CKD TRANSITION

(71) Applicant: Northwestern University, Evanston, IL (US)

(72) Inventors: Pinelopi Kapitsinou, Evanston, IL (US); Ratnakar Tiwari, Evanston, IL (US)

(21) Appl. No.: 18/420,725

(22) Filed: Jan. 23, 2024

Related U.S. Application Data
(60) Provisional application No. 63/481,180, filed on Jan. 23, 2023.

(51) Int. Cl. A61K 31/4375; A61P 13/12

(52) U.S. Cl. CPC A61K 31/4375; A61P 13/12

(57) ABSTRACT

Disclosed are agents, compositions, and methods for treating acute kidney injury (AKI). Particularly disclosed are agents, compositions, and methods for treating AKI and reducing the progression from AKI to chronic kidney disease by administering an agent that inhibits or reduces expression of monocarboxylate transporter 4 (MCT4).

### CROSS-REFERENCE TO RELATED APPLICATIONS

This application is related to, claims priority to, and incorporates herein by reference for all purposes U.S. Provisional Patent Application No. 63/481,180, filed Jan. 23, 2023.

### STATEMENT REGARDING FEDERALLY SPONSORED RESEARCH

This invention was made with government support under grant number DK115850 awarded by the National Institutes of Health. The government has certain rights in the invention.

### REFERENCE TO AN ELECTRONIC SEQUENCE LISTING

The contents of the electronic sequence listing (70258102461_SL_ST26.xml; Size: 16,737 bytes; and Date of Creation: Jan. 19, 2024) is herein incorporated by reference in its entirety.

### FIELD OF THE INVENTION

The disclosed technology is generally directed to treatment of acute kidney injury. More particularly the technology is directed to treatment of acute kidney injury to decrease the transition to chronic kidney disease.

### BACKGROUND

Globally, AKI affects over 13 million people and results in approximately 1.7 million deaths each year. Those who survive AKI have a higher risk for later development of chronic kidney disease (CKD). Treatments currently available are dialysis and kidney transplant, both of which are associated with shortened life expectancy. AKI and CKD impose a significant burden on society. Therefore, there is a need for improved medical treatments for AKI and for slowing the transition from AKI to CKD.

### SUMMARY

Disclosed are agents, compositions, and methods for treating acute kidney injury (AKI). Particularly disclosed are agents, compositions, and methods for treating AKI and reducing the progression from AKI to chronic kidney disease by administering an agent that inhibits or reduces expression of monocarboxylate transporter 4 (MCT4).

Another aspect of the technology provides for a method for treating fibrosis in a subject in need thereof. The method may comprise administering an agent that inhibits or reduces expression of MCT4.

Another aspect of the technology provides for a method for treating inflammation in a subject in need thereof. The method may comprise administering an agent that inhibits or reduces expression of MCT4.

### BRIEF DESCRIPTION OF THE DRAWINGS

The patent or application file contains at least one drawing executed in color. Copies of this patent or patent application publication with color drawings will be provided by the Office upon request and payment of the necessary fee.

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention.

FIG. 1. Post-ischemic administration of the MCT4 inhibitor syrosingopine in AKI to CKD transition in wild type mice. (A) Schematic view of experimental protocol applied for data shown in FIGS. 2 &3. (B) Representative images of Day 14 post-ischemic kidneys of wild type mice treated with vehicle (WT+Veh) or syrosingopine (WT+Syro). Image of uninjured kidney is shown for reference.

FIG. 2. Post-ischemic treatment with the MCT4 inhibitor syrosingopine ameliorates AKI to CKD transition in wild type mice following kidney IRI. Top: Shown are representative images of H&E- and Picro-Sirius red-stained sections from day 7 and day 14 post-IRI kidneys of mice treated with vehicle (Veh) or syrosingopine (Syro). Baseline conditions (no injury) are shown for comparison. Bottoms panels show tubular injury scores, quantification of Picro Sirius Red^{+ve} area and the expression levels of selected profibrotic and pro-inflammatory genes in the indicated experimental groups.

FIG. 3. Post-ischemic treatment with the MCT4 inhibitor syrosingopine significantly alters the kidney transcriptome in AKI to CKD transition induced by IRI in wild type mice. Bulk RNA-seq analysis was performed on day 7 and day 14 post-ischemic kidneys from syrosingopine and vehicle treated mice. Shown are bubble charts for top 10 enriched Hallmark pathways of upregulated or downregulated DEGs by Syrosingopine. (A) VB7 vs ST7, Top 10 down; (B) ST14 vs VD14, Top 10 up. (C) ST14 vs VD14, Top 10 down. Note that at Day 7 post IRI no significantly upregulated pathways were detected. VB7; vehicle treated day 7 post-ischemic kidneys, ST7; syrosingopine treated day 7 post-ischemic kidneys; VB14; vehicle treated day 14 post-ischemic kidneys, ST14; syrosingopine treated day 14 post-ischemic kidneys.

FIG. 4. Post-ischemic inactivation of endothelial PHD2 does not alter post-ischemic kidney injury. (A) Experimental scheme illustrates the timing of unilateral renal artery clamping, tamoxifen administration and analysis. (B) Representative images of H&E and Picro-Sirius red stained sections from day 14 post-ischemic kidneys of PHD2^{iEC} mutants and their Cre⁻ littermates. Right panels show tubular injury score (Top) and semi-quantitative analysis of Picro-Sirius red^{+ve} area in the indicated genotypes (Cre-left, PHD2^{iEC} right). Scale bars indicate 100 m and 200 m for H&E and Picro-Sirius red images, respectively. (C) mRNA levels of Loxl2, Tgfb1 and Acta2 in IR and CTL kidneys from PHD2^{iEC} mice and their Cre⁻ controls at day 14 after unilateral IRI. Circles show Cre- (left) and PHD2^{IEC} (right) control data. Squares correspond to Cre- IR (left) and PHD2mc IR (right). All bars show mean±SEM. For (B), unpaired t-test with Welch's correction was used. For (C), statistics were determined using one-way ANOVA with Sidak correction for multiple comparisons. *, P<0.05; ns, not statistically significant. uIRI, unilateral ischemia reperfusion injury; CTL, contralateral; IR, kidney subjected to ulRI; Rel., relative.

FIG. 5. scRNA-seq analysis shows differential expression of PHD1, PHD2 and PHD3 in kidney ECs in mouse and humans. (A-C) scRNA-seq analysis of RECs extracted from mouse EC Atlas database (https://endotheliomics.shinyapps.io/ec_atlas/). (A) Uniform manifold approximation and projection (UMAP) plot shows three EC clusters; cortical RECs (cRECs), medullary RECs (mRECs) and glomerular RECs (gRECs). Dot plot displays gene expression patterns of cluster-enriched markers. Violin plots (B) and feature plots (C) show the expression of Phd1 (Egln2), Phd2 (Egln1) and Phd3 (Egln3) in murine RECs. (D-F) scRNA-seq analysis of RECs extracted from normal human kidney biopsies (n=24). (D) UMAP plot shows arteriolar RECs (aRECs), glomerular RECs (gRECs) and peritubular RECs (pRECs). Dot plot illustrates gene expression patterns of cluster-enriched markers. Violin plots (E) and feature plots (F) show the expression of PHDs in different RECs clusters.

FIG. 6. Post-ischemic simultaneous inactivation of endothelial PHD1, 2, and 3 promotes maladaptive kidney repair. (A) Scheme illustrating the experimental strategy applied for unilateral renal IRI (uIRI) studies. PHD^{TiEC} mice and their Cre⁻ littermates were subjected to 25 minutes of unilateral renal artery clamping. Treatment with tamoxifen was started on day 1 post uIRI involving 4 IP doses given every other day. Mice were sacrificed for histopathological and molecular analysis on day 14 post uIRI. (B) Representative images of H&E and Picro-Sirius red stained sections as well as tubular injury score and semi-quantitative analysis of Picro-Sirius red^{+ve} area on day 14 post-ischemic kidneys from PHD^{TiEC} mice and Cre⁻ littermates. Scale bars indicate 100 μm and 200 μm for H&E and Picro-Sirius red images, respectively. Right panels show injury score (top) and Picro-Sirius^{+ve} area % (bottom) for Cre- (left) and PHD^{IEC} (right). (C) mRNA levels of Loxl2, Tgfb1 andActa2 in IR and CTL kidneys from PHD^{TiEC} mice or their Cre⁻ controls at day 14 after uIRI. Circles show control data for Cre- (left) and PHD^{IEC} (right). Squares correspond to Cre- IR (left) and PHD2^{IEC} IR (right). (D) Representative images of EMCN immunostaining and semiquantitative analysis of EMCN^{+ve} peritubular capillary area on day 14 post-ischemic kidneys from PHD^{TiEC} mice and Cre⁻ littermates. Right panels show EMCN^{+ve} area for Cre- (left) and PHD^{IEC} (right). (E) Scheme depicting the experimental workflow for bilateral renal IRI (bIRI) studies. PHD^{TiEC} mice and their Cre⁻ littermates were subjected to 23 minutes of bilateral renal artery clamping followed by tamoxifen treatment as described in A. Serum BUN levels were measured on 1 day prior to bIRI (baseline) and on days 1, 7 and 14 post bIRI. GFR was measured by FITC-sinistrin clearance on day 14 post bIRI using the MediBeacon transdermal GFR monitor system. (F) Serum BUN levels at different time points and GFR measurements on day 14 post bIRI. All bars show mean±SEM of each group (n=4-8), Cre- (left) and PHD^{IEC} (right). For (B), (D) and (F), statistics were determined by unpaired t-test with Welch's correction. For (C), one-way ANOVA with Sidak correction for multiple comparisons was used. *, P<0.05; **, P<0.01; ns, not statistically significant. CTL, contralateral kidney; IR, kidney subjected to IRI; bIRI, bilateral IRI; Rel., relative.

FIG. 7. scRNA-seq analysis reveals the cellular landscape of day 14 post-ischemic kidneys of PHD^{TiEC} and control mice. (A) Scheme illustrating the experimental strategy applied for scRNA-seq analysis. A PHD^{TiEC} mouse and a Cre⁻ littermate were subjected to 25 minutes of unilateral renal artery clamping. Treatment with tamoxifen was started on day 1 post uIRI and involved 4 IP doses given every other day. Mice were sacrificed for scRNA-seq analysis on day 14 post uIRI. IR kidneys were isolated and single cell suspension was prepared and used for scRNA-seq analysis (n=1 per genotype). (B) Uniform manifold approximation and projection (UMAP) plot representation of the cell classification in day 14 post-ischemic kidneys from PHD^{TiEC} and Cre⁻ mice. (C) Violin plots display characteristic marker genes for each identified cell population. Right side bar plot shows cell proportions in day 14 post-ischemic kidneys from Cre⁻ and PHD^{TiEC} mice. Highlighted is the increased proportion of fibroblasts (FIB) in PHD^{TiEC} post-ischemic kidney compared to Cre⁻ control. Proximal tubule (PT), Injured proximal tubule (Injured-PT), Thick ascending limb (TAL), Distal convoluted tubule (DCT), Collecting duct (CD), Proliferating CD (pCD), Inner medullary collecting duct (IM-CD), Collecting duct-intercalated cells (CD-IC), Intercalated cells (IC), Parietal cells (PAR), Fibroblasts (FIB), Pericytes (PER), Endothelial cells 1-3 (EC1-3), Urothelial cells (URO), Macrophages 1-4 (Mφ1-4), Proliferating macrophages (pMφ), C1q (C1qa, C1qb and C1qc) expressing immune cells (C1q-IM), T cells (T), Proliferating T cells (pT), Natural killer cells (NK), B cells (B), Dendritic cells (DEN), and Neutrophils (NEU). (D) Top 2 enriched Hallmark pathways emerged in GSEA (gene set enrichment analysis) hallmark analysis of differentially expressed genes (DEGs) for PT, DCT and CD clusters of PHD^{TiEC} post-ischemic kidney as compared to Cre⁻ control.

FIG. 8. Post-ischemic endothelial PHD inactivation induces a hypoxia and glycolysis gene signature in mRECs. (A) Dot plot visualization shows the expression of marker genes used to identify cRECs, mRECs and EndMT-RECs clusters. (B) GSEA in mRECs of PHD^{TiEC} kidney compared to control. Among the most highly enriched Hallmark pathways were Hypoxia and Glycolysis. (C) Violin plots show significantly upregulated glycolytic genes in mRECs of PHD^{TiEC} compared to control. Pathway diagram summarizes the functions of up-regulated genes (Slc2a1, Gpi1, Aldoa, Tpi1, Pgk1, Eno1, Pkm, Ldha, Slc16a3) in glycolysis. (D-E) snRNA-seq analysis of human kidney tissue from patients with severe AKI and controls (n=6-8). Analysis was performed on publicly available snRNA-seq data from Christian Hinze et al [Hinze C, et al., Single-cell transcriptomics reveals common epithelial response patterns in human acute kidney injury.

Genome Med. 2022;14(1):103]. (D) Bubble chart for top 10 enriched Hallmark pathways of upregulated DEGs in kidney ECs from patients with severe AKI compared to controls. (E) Box plots show the expression of glycolytic genes in kidney ECs in controls vs AKI patients. The expression levels of glycolytic genes were extracted from the online interface provided by Christian Hinze et al (https://shiny.mdc-berlin.de/humAKI). CPM, normalized counts per million; cRECs, cortical renal ECs; mRECs, medullary renal ECs; EndMT-RECs, renal ECs expressing mesenchymal markers.

FIG. 9. Post-ischemic inactivation of endothelial PHDs induces EC derived pro-inflammatory responses. (A) GSEA plots show significant enrichment for GO-biological processes (GOBP) of leukocyte migration and myeloid leukocyte migration in mRECs of PHD^{TiEC} post-ischemic kidney compared to control. (B) Violin plots display the expression levels of pro-inflammatory genes associated with core enrichment in GOBP-leukocyte migration and myeloid leukocyte migration in mRECs of PHD^{TiEC} compared to control. (C) Shown is the experimental strategy for flow cytometry analysis. Eight days after uIRI, post-ischemic kidneys from PHD^{TiEC} and Cre⁻ control mice were harvested, and flow cytometry analysis of immune cells was performed (n=4-5). Data are represented as mean±SEM. Statistics were determined by unpaired t-test with Welch's correction. (D) Representative images of immunofluorescence staining for F4/80 (green) and nuclear DAPI staining (blue) of day 8 post-ischemic kidneys from PHD^{TiEC} and Cre control mice. Images were captured using a Nikon Ti2 Widefield fluorescence microscope. Scale bar indicates 100 m. (E) Shown is NicheNet analysis of mRECs communication with macrophage Mφ1 cluster in day 14 post-IRI kidney of PHD^{TiEC} mutant compared to control. Top prioritized ligands expressed by mRECs (senders) and target genes that are significantly altered (red, up-regulated genes; blue, down-regulated genes) in the macrophages Mφ1 (receivers). The interaction pairs were derived from the NicheNet data sources and analysis. (F) Hallmark analysis of significantly upregulated genes in Mφ1 cluster of PHD^{TiEC} kidney compared to Cre control. Top 10 pathways are shown. *, P<0.05; ns, not statistically significant.

FIG. 10. Post-IRI treatment with the MCT4 inhibitor syrosingopine restores adaptive kidney repair in PHD^{TiEC} mice. (A) Representative images of immunofluorescence staining for MCT4 (red) and EMCN (green) of contralateral and day 14 post-ischemic kidneys from PHD^{TiEC} mice. Zoom-in panels show the increased expression of MCT4 in EMCN^{+ve} cells in PHD^{TiEC} post-ischemic kidney. Images were captured using a Nikon Ti2 Widefield fluorescence microscope. Scale bar, 100 μm. (B) Scheme shows the experimental protocol used. PHD^{TiEC} mice were subjected to 25 minutes of unilateral renal artery clamping. Tamoxifen was started on day 1 post uIRI and was given every other day until day 7 post uIRI. Treatment with syrosingopine was started at day 2 post uIRI and was given every other day until day 14, when mice were sacrificed for histopathological and molecular analysis. (C) Representative images of uninjured kidney compared to day 14 post-ischemic kidneys treated with vehicle, or syrosingopine. All mice are PHD^{TiEC} mutants. (D) Representative images of H&E and Picro-Sirius red stained day 14 post-ischemic kidneys from vehicle- vs syrosingopine-treated PHD^{TiEC} mutants. Right: Tubular injury score and semiquantitative analysis of Picro-Sirius red^{+ve} area of day 14 post-ischemic kidneys for the indicated experimental groups. Scale bars indicate 100 m and 200 m for H&E and Picro-Sirius red images, respectively. (E) mRNA levels of Loxl2, Tgfb1 andActa2 in CTL and IR kidneys from vehicle or syrosyngopine treated PHD^{TiEC} mice on day 14 after uIRI. Data are represented as mean±SEM. For (D), statistics were determined by unpaired t-test with Welch's correction. For (E), statistics were determined using one-way ANOVA with Sidak correction for multiple comparisons. n=4-5; *, P<0.05; ***, P<0.001. uIRI, unilateral IRI; CTL, contralateral kidney; IR, kidney subjected to IRI; veh, vehicle; Syro, syrosingopine.

FIG. 11. Syrosingopine or MCT4 knockdown suppresses the expression of EC adhesion molecules in HPAECs activated by Hypoxia/Reoxygenation and IL-1β. (A) Experimental scheme for HPAECs subjected to 0.5% O₂ for 18 hours in the presence of syrosingopine (5 PM) or MCT4 siRNA followed by reoxygenation for 8 hours in the presence of IL-10 (1 ng/ml). (B) mRNA levels of VCAM1 and ICAM1 in syrosingopine- vs vehicle-treated HPAECs, that were activated by Hypoxia/Reoxygenation and IL-10. (C) THP1 monocyte adhesion to inflamed ECs. THP1 monocyte cells, labeled with Green CMFDA Dye, were introduced on a monolayer of HPAECs that had been subjected to the indicated experimental conditions. Following a 90-minute incubation period, floating cells were washed away and adhered THP1 cells were visualized using a fluorescent microscope and subsequently quantified. Representative images of fluorescent THP1 cells attached to ECs in different experimental groups are presented. Scale bar, 200 m. (D) mRNA expression of VCAM1 and ICAM1 in HPAECs transfected with control or MCT4 siRNA and subjected to Hypoxia/Reoxygenation and IL-10. (E) THP1 monocyte adhesion to inflamed ECs. Labeled THP1 cells were introduced on a monolayer of HPAECs that had been subjected to the indicated experimental conditions. Following incubation, adhered THP1 cells were visualized quantified as in C. Representative images of fluorescent THP1 cells attached to ECs in different experimental groups are presented. Scale bar, 200 m. Data are represented as mean±SEM. Statistics were determined by one-way ANOVA with Sidak correction for multiple comparisons. n=3-4; *, P<0.05; **, P<0.01; ***, P<0.001; ****P<0.0001; ns, not statistically significant. Nx, Normoxia; Hx, Hypoxia/Reoxygenation; Veh, vehicle; Syro, syrosingopine; C, negative control siRNA; MCT4si, MCT4siRNA.

FIG. 12. Efficiency of Cre- recombination in Cdh5(PAC)-CreER^{T2} transgenic mice following kidney IRI. (A) By crossing Cdh5(PAC)-CreER^{T2} mice with ROSA26-ACTB-tdTomato, −EGFP (mTmG) reporter mice, we generated Cdh5(PAC)-CreER^{T2}; Rosa26-mTmG mice, in which successful Cre- mediated excision is being indicated by GFP expression in endothelial cells (ECs). Mice were subjected to uIRI followed by tamoxifen administration starting at day 1 post uIRI to a total of 4 intraperitoneal (IP) injections given every other day. The degree of EC-specific recombination was assessed by FACs analysis in single-cell suspensions prepared by CTL and IR kidneys. (B) Flow cytometric gating strategy used to define recombined ECs. Staining for the endothelial marker CD31 was used to detect ECs, while GFP identified cells with tamoxifen induced expression of Cdh5-CreER^{T2}. (C) Shown are the percentages of GFP^{+ve} and CD31^{+ve} cells (left panel) as well as the percentage of CD31^{+ve} cells among GFP^{+ve} cells (right panel) in CTL and IR kidneys at day 14 post uIRI (n=3). For (C), statistics were determined by one-way ANOVA with Sidak correction for multiple comparisons. ns, not statistically significant. ECs, endothelial cells.

FIG. 13. Murine kidney endothelium shows compartment-specific differences in the expression of PHD1, PHD2 and PHD3. Schematic view of experiment and representative images of immunofluorescence staining for PHD1, PHD2, PHD3 (magenta) and nuclear DAPI staining (blue) on kidney sections from Cdh5(PAC)-CreER^{T2}-Rosa26mTmG reporter mice after tamoxifen-induced recombination. Non recombined cells express membrane bound mTomato (red), whereas recombined cells express membrane-bound EGFP (green).

FIG. 14. Simultaneous acute inactivation of endothelial PHD1, 2, and 3 stabilizes HIF without significantly affecting kidney morphology. (A) Schematic diagram depicting the tamoxifen administration regimen and the experimental strategy to obtain PHDTiEC mice and Cre- control mice. (B) Immunofluorescence staining for PHD1-3 (red), EMCN (green) and nuclear DAPI (blue) using kidney sections from PHD^{TiEC} mice and Cre- controls collected 1 week after the last dose of tamoxifen. Scale bar, 100 m. (C) Immunoblot analysis of HIF-1a and HIF-2a in kidney nuclear extracts isolated from PHD^{TiEC} mice and Cre- littermates. Co, positive control. (D) Representative images of H&E and Picro-Sirius red stained kidney sections for the indicated genotypes(Cre- left, PHD^{TiEC} right). Right side graph shows semi-quantitative analysis of Picro-Sirius red^{+ve} area for PHD^{TiEC} mice (right) and Cre- littermates (left) 1 week after the last dose of tamoxifen (n=4-6). Scale bars indicate 100 m and 200 m for H&E and Picro-Sirius red images, respectively. Data are represented as mean±SEM. Statistics were determined by unpaired t-test with Welch's correction. *, P<0.05; ns, not statistically significant. EMCN, endomucin.

FIG. 15. scRNA-seq analysis showing similar cell populations in day 14 post-ischemic kidneys of PHD^{TiEC} and Cre- mice. (A) UMAP showing different cell clusters in day 14 post-ischemic kidneys from PHD^{TiEC} and Cre- mice. (B) UMAP after overlaying samples of Cre- and PHD^{TiEC} day 14 post-ischemic kidneys showing similar clustering.

FIG. 16. Marker genes used to identify different EC clusters. (A) UMAP plot with highlighted EC1 (red), EC2 (green) and EC3 (blue) cluster. (B) Feature plot showing expression of endothelial marker genes Cd34 and Pecam1 in EC1, EC2 and EC3 clusters. (C) Feature plot of EC marker genes used to identify mRECs (EC1), cRECs (EC2) and EndMT-RECs (EC3) clusters.

FIG. 17. Violin plots showing the expression of endothelial and mesenchymal markers in EC1, EC2 and EC3 clusters. Violin plots show the expression of marker genes for ECs (Cd34, Pecam1, Cldn5) and mesenchymal cells (Vim, Cald1, Fn1, Acta2, Myl9, Myh11, Tagln, Tpm2, Mustn1, and Mmp2) in three EC clusters.

FIG. 18. Strategy for the analysis of immune cells by flow cytometry. Schematic view of FACs experiment and gating strategy for the analysis of different immune cell populations in day 8 post-IRI kidneys from PHD^{TiEC} and Cre- mice. CD45+ (leukocytes), CD45+ CD3+ (T cells), CD45+ CD11b+ (myeloid cells), CD45+ CD11b+ F4/80+ (macrophages), CD45+ CD11b+ Ly6C+ (monocytes), CD45+ CD11b+ Ly6G+ (granulocytes) cells were measured.

FIG. 19. MCT4 expression in contralateral and day 14 post-ischemic kidney of PHD^{TiEC} mouse. Representative images of immunofluorescence staining for MCT4 (red) and EMCN (green) of CTL and IR kidneys on day 14 post-IRI from PHD^{TiEC} mice indicating increased expression of endothelial MCT4 following ischemic injury. Images were captured using Nikon a Ti2 Widefield fluorescence microscope. Scale bar, 100 μm. CTL, contralateral kidney; IR, kidney subjected to uIRI.

FIG. 20. Effect of syrosingopine on extracellular lactate levels and knockdown efficiency of MCT4 siRNA. (A) HPAECs were treated for 24 hours with syrosingopine or vehicle (DMSO) and extracellular lactate levels were measured. (B) Experimental scheme for HPAECs subjected to 0.5% O2 for 18 hours in the presence of MCT4 siRNA followed by reoxygenation for 8 hours in the presence of IL-10 (1 ng/ml). Graph showing SLC16A3 (MCT4) mRNA levels in cells transfected with MCT4 siRNA compared to negative control siRNA transfected cells. All bars show mean±SEM. For (A), unpaired t-test with Welch's correction was used. For (B), statistics were determined using one-way ANOVA with Sidak correction for multiple comparisons. *, P<0.05; ***, P<0.001; ****, P<0.0001. Veh, vehicle; Syro, syrosingopine; Nx, Normoxia; Hx, Hypoxia/Reoxygenation; C, negative control siRNA; MCT4si, MCT4 siRNA.

### DETAILED DESCRIPTION

Described here are agents and methods for treating acute kidney injury (AKI) and subsequent transition to chronic kidney disease (CKD) in a subject in need thereof by targeting monocarboxylate transporter 4 (MCT4). More specifically, disclosed herein is an agent that inhibits MCT4 or reduces expression of MCT4.

Monocarboxylate transporters, or MCTs, are a family of proton-linked plasma membrane transporters that carry molecules having one carboxylate group such as lactate, pyruvate, and ketones across biological membranes. There are 14 known MCTs and they are expressed in nearly every kind of cell. MCT4 is encoded by the solute carrier family 16 member 3 (SLC16A3) gene. MCT4 demonstrates a high affinity for lactate over other monocarboxylates. MCT4 forms a heterodimeric complexes with the cell surface glycoprotein CD147 and is essential for maintaining lactate and pH homeostasis.

As used herein, the term “agents” can include MCT4 inhibitors as well as compositions that affect the expression of MCT4. A person skilled in the art will understand that the term “inhibitor”, as used herein, includes compounds that can decrease or block the action or function of the monocarboxylate transporter 4 protein. The term “inhibiting MCT4 activity” as used herein, can refer to decreasing the transport activity of MCT4. An agent of the present invention can hinder or slow MCT4 activity, e.g., transport activity with regard to lactate or H⁺ efflux.

The agent can be a composition that reduce the expression of the SLC16A3 gene, transcription of SLC16A3 to mRNA, or the translation of the MCT4 mRNA. The agent may be RNA-based interference agent, such as a siRNA, shRNA, or miRNA, that can target mRNA and block translation. The agent may also be a CRISPR-mediated gene knockdown agent, such as dCas9, that can bind to DNA block transcription or Cas13 that can be used for RNA knockdown.

As used herein, a “subject” maybe be interchangeable with “patient” or “individual” and means an animal, which may be a human or non-human animal, in need of treatment. In particular embodiments, the subject is a human subject.

A “subject in need of treatment” may include a subject having a disease, disorder, or condition that may be characterized by a kidney disease or disorder, fibrosis in the kidney, or inflammation associated with or that contributes to the development or progression of a kidney disease or disorder. A subject in need of treatment may have elevated levels of profibrotic proteins or proinflammatory proteins.

The subject in need of treatment may have AKI or CKD. AKI can be caused by lack of blood flow to the kidneys, blockage in urine flow that causes infections, or direct kidney damage by infections, medications, toxins, or autoimmune conditions. AKI can be a community-acquired disease in rural areas, where specific causes may include diarrheal diseases with dehydration, infectious disease (e.g. malaria), animal bites, venomous insect bites, or sepsis. In urban areas, AKI can afflict hospitalized patients, especially older adults. Diseases and conditions that lead to AKI include hypotension, ischemia-reperfusion (e.g., vascular surgeries and kidney transplantation), heart failure, decreased heart function, overuse of NSAID pain medicines, and certain cancers of the bladder, prostate, or cervix.

Those who survive AKI have a higher risk for later development of CKD. CKD is caused by the progressive loss of structure or function of kidneys which manifests in symptoms of swelling, fatigue, shortness of breath, confusion, nausea, seizures or coma, or chest pain. CKD involves a gradual loss of kidney function that can progress to end-stage kidney failure, which is fatal without artificial filtering (dialysis) or a kidney transplant. Inflammation has been recognized as an important component of CKD. A common pathway in CKD is fibrosis, or the formation of internal scar tissue, that can ultimately lead to end-stage kidney failure.

Medications for AKI and CKD include angiotensin-converting enzyme (ACE) inhibitors or angiotensin II receptor blockers (ARBs) to lower blood pressure and diuretics to remove extra fluid. A therapeutic intervention that has an established beneficial effect in the management of AKI is the intravenous (IV) administration of isotonic sodium chloride solution. Other treatments currently available are dialysis and kidney transplant.

Methods for treating subjects with the agents disclosed herein are provided. Suitably, the methods for treating a subject can include administering to the subject an effective amount of one or more agents than can inhibit MCT4 or reduce expression of MCT4. The methods for treating a subject can include administering to the subject a pharmaceutical composition comprising the effective amount of one or more agents that inhibit MCT4 or reduce expression of MCT4. The methods for treating a subject can include co-administering to the subject an effective amount of one or more agents than can inhibit MCT4 or reduce expression of MCT4 along with another modality of treatment. Such modalities can include currently accepted treatments for AKI or CKD as described herein.

As used herein, the terms “treating” or “to treat” each mean to alleviate symptoms, eliminate the causation of resultant symptoms either on a temporary or permanent basis and/or to prevent or slow the appearance or to reverse the progression or severity of resultant symptoms of the named disease or disorder. As such, the methods disclosed herein encompass both therapeutic and prophylactic administration. In some embodiments, the subject is responsive to therapy with one or more of the agents disclosed herein in combination with one or more additional therapeutic agents.

As used herein the term “effective amount” refers to the amount or dose of the agent that provides the desired effect. In some embodiments, the effective amount is the amount or dose of the agent, upon single or multiple dose administration to the subject, which provides the desired effect in the subject under diagnosis or treatment.

An effective amount can be readily determined by those of skill in the art, including an attending diagnostician, by the use of known techniques and by observing results obtained under analogous circumstances. In determining the effective amount or dose of agent administered, a number of factors can be considered by the attending diagnostician, such as: the species of the subject; its size, age, and general health; the degree of involvement or the severity of the disease or disorder involved; the response of the individual subject; the particular agent administered; the mode of administration; the bioavailability characteristics of the preparation administered; the dose regimen selected; the use of concomitant medication; and other relevant circumstances.

Agents may be administered to a subject in an effective amount in such that the one or more isoforms of MCT4 is inhibited or expression of the one or more isoforms of MCT4 is reduced. Suitably, the desired effects of the agents may include slowing transport by MCT4, halting transport by MCT4, slowing fibrosis, halting fibrosis, reversing fibrosis, reducing inflammation, improving perfusion, or combinations thereof. In some embodiments, the effective amount of the agent results in reducing the expression of proteins associated with fibrosis and/or inflammation (profibrotic or proinflammatory proteins) or increasing the expression of anti-fibrotic proteins or anti-inflammatory proteins. In particular embodiments, the effective amount of the agent results in reducing the mRNA levels of at least one of Loxl2, Tgfβ1, Acta2, Vcam1, or Icam1.

Other inhibitors of MCT4 may include but are not limited to 5-{2-[5-Chloro-2-(5-ethoxyquinoline-8-sulfonamido)phenyl]ethynyl}-4-methoxypyridine-2-carboxylic acid [Heinrich T., et al. J. Med. Chem. 2021, 64, 16, 11904-11933], AZD0095 [Goldberg, F. W. et al. J. Med. Chem. 2023, 66, 1, 384-397], or VB124 [Cluntun, A. A. et al. Cell Metabolism 2021, 33, 629-648]. Agents may be used singularly or in combination with two, three, four, or more than four other agents.

In one embodiment, the agent can be a derivative of reserpine. In a particular embodiment, the agent can be syrosingopine. Syrosingopine (CAS No.: 84-36-6) is a reversible inhibitor of MCT4 transport activity. Syrosingopine is an anti-hypertensive drug, and is a dual inhibitor, inhibiting both MCT1 and MCT4, while exhibiting a 60-fold higher potency towards MCT4.

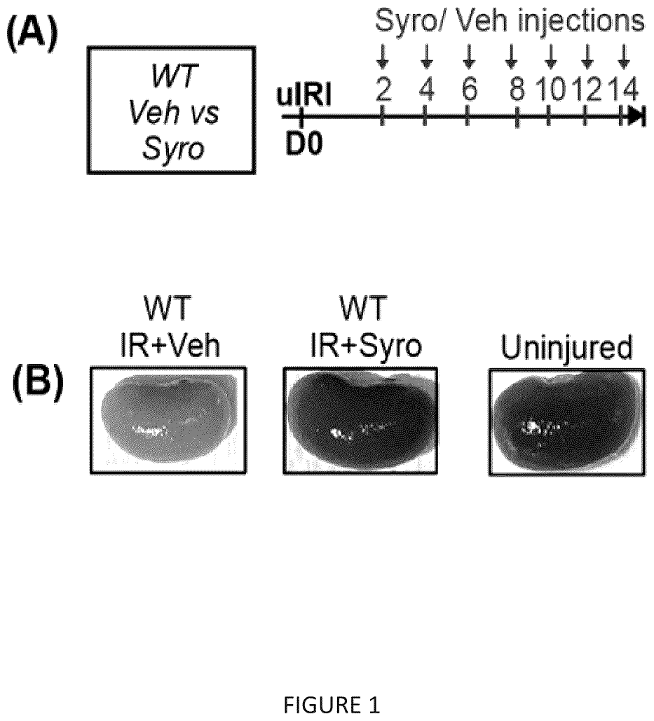

As shown in the Examples and FIGS. 1-3, syrosingopine is effective for improving perfusion in kidneys, reducing fibrosis and inflammation. FIG. 1A shows a schedule for therapeutic administration of syrosingopine after AKI. The result of administering syrosingopine is improved perfusion of blood in the injured kidney, as evidenced by the darker coloration compared to the injured untreated kidney (FIG. 1B).

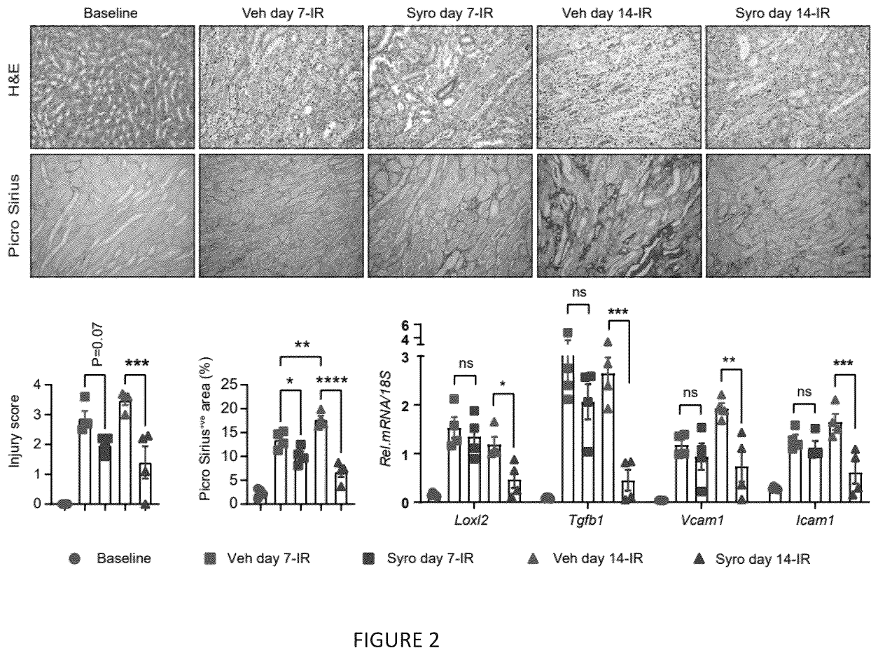

Fibrosis is reduced in the syrosingopine-treated kidneys as evidenced by histological staining (FIG. 2). The stain, Picro-Sirius red is a widely used histological technique to visualize the distribution of collagen in fibrotic tissue sections by bright-field or polarization microscopy. Image analysis of the stained areas indicates that the samples from the syrosingopine-treated kidneys (FIG. 2, bottom) show significantly less staining, and therefore less fibrosis, than the samples from the vehicle-treated controls at day 7 and day 14 post IRI. Finally, tubular injury scoring shows less injury in syrosingopine-treated mice compared to controls (FIG. 2).

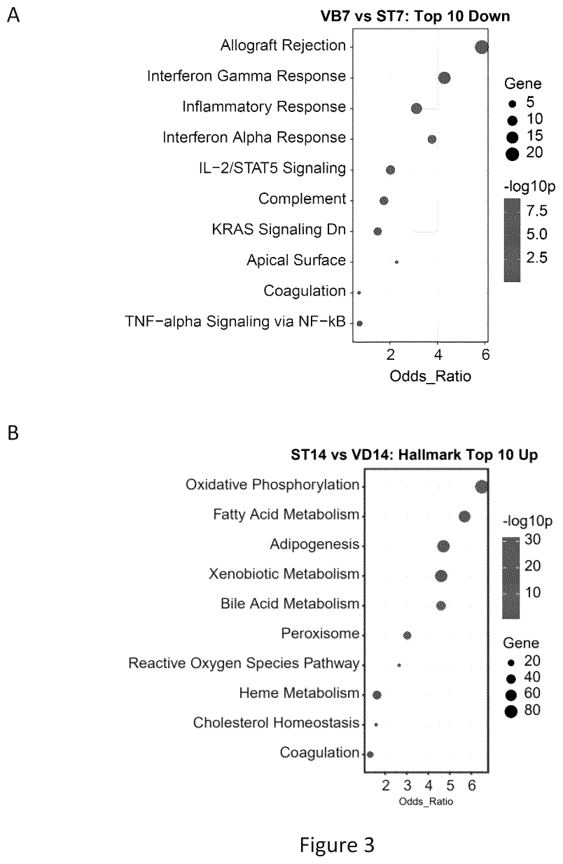

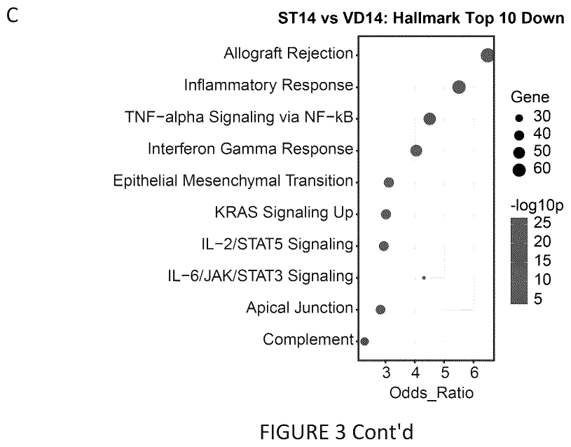

The effect of syrosingopine on gene expression was examined (FIG. 2 bottom). RNA was extracted from vehicle-treated and syrosingopine-treated injured kidneys at two time points post IRI, day 7 and day 14. After reverse transcription, qPCR analysis showed that syrosingopine-treated injured kidneys show substantially lower levels of profibrotic mRNAs lysyl oxidase-like 2 (Loxl2) and transforming growth factor-beta 1 (Tgfβ1) as well as pro-inflammatory transcripts vascular cell adhesion molecule 1 (Vcam1), and intercellular cell adhesion molecule 1 (Icam1) compared to the vehicle treated controls. Consistent were the results of the unbiased bulk RNA-seq analysis shown in FIG. 3. Hallmark analysis of the differentially expressed genes (DEGs) showed suppression of pro-inflammatory pathways in day 7 and day 14 post-IRI kidneys of mice treated with syrosingopine compared to vehicle treated controls (FIGS. 3A and 3C). Furthermore, metabolic pathways important for tubular health and function, such as oxidative phosphorylation and fatty acid metabolism, were significantly up-regulated in day 14 post-IRI kidneys of mice treated with syrosingopine compared to controls (FIG. 3B). Therefore, treatment with syrosingopine reduces fibrosis and inflammation and preserves metabolic health of injured kidney tissue.

The agents disclosed herein may be formulated as pharmaceutical compositions that include: an effective amount of one or more agent, and one or more pharmaceutically acceptable carriers, excipients, or diluents. Where a substance or compound is used the enhance permeability of the agent, the substance or compound may be included in the pharmaceutical composition comprising the agent or provided in a separate pharmaceutical composition.

The pharmaceutical composition may include the agent in a range of about 0.1 to 2000 mg (preferably about 0.5 to 500 mg, and more preferably about 1 to 100 mg). The pharmaceutical composition may be administered to provide the agent at a daily dose of about 0.1 to 100 mg/kg body weight (preferably about 0.5 to 20 mg/kg body weight, more preferably about 0.1 to 10 mg/kg body weight). In some embodiments, after the pharmaceutical composition is administered to a patient (e.g., after about 1, 2, 3, 4, 5, or 6 hours post-administration), the concentration of the agent at the site of action is about 2 to 10 mM.

The agents utilized in the methods disclosed herein may be formulated as a pharmaceutical composition in solid dosage form, although any pharmaceutically acceptable dosage form can be utilized. Exemplary solid dosage forms include, but are not limited to, tablets, capsules, sachets, lozenges, powders, pills, or granules, and the solid dosage form can be, for example, a fast melt dosage form, controlled release dosage form, lyophilized dosage form, delayed release dosage form, extended-release dosage form, pulsatile release dosage form, mixed immediate release and controlled release dosage form, or a combination thereof.

The agents utilized in the methods disclosed herein may be formulated as a pharmaceutical composition that includes a carrier. For example, the carrier may be selected from the group consisting of proteins, carbohydrates, sugar, talc, magnesium stearate, cellulose, calcium carbonate, and starch-gelatin paste.

The agents utilized in the methods disclosed herein may be formulated as a pharmaceutical composition that includes one or more binding agents, filling agents, lubricating agents, suspending agents, sweeteners, flavoring agents, preservatives, buffers, wetting agents, disintegrants, and effervescent agents. Suitable diluents may include pharmaceutically acceptable inert fillers.

The agents utilized in the methods disclosed herein may be formulated as a pharmaceutical composition for delivery via any suitable route. For example, the pharmaceutical composition may be administered via oral, intravenous, intramuscular, subcutaneous, topical, and pulmonary route.

Examples of pharmaceutical compositions for oral administration include capsules, syrups, concentrates, powders and granules.

The agents utilized in the methods disclosed herein may be administered in conventional dosage forms prepared by combining the active ingredient with standard pharmaceutical carriers or diluents according to conventional procedures well known in the art. These procedures may involve mixing, granulating and compressing or dissolving the ingredients as appropriate to the desired preparation.

Pharmaceutical compositions comprising the agents may be adapted for administration by any appropriate route, for example by the oral (including buccal or sublingual), rectal, nasal, topical (including buccal, sublingual or transdermal), vaginal or parenteral (including subcutaneous, intramuscular, intravenous or intradermal) route. Such formulations may be prepared by any method known in the art of pharmacy, for example by bringing into association the active ingredient with the carrier(s) or excipient(s).

The formulations may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets.

The agents employed in the compositions and methods disclosed herein may be administered as pharmaceutical compositions and, therefore, pharmaceutical compositions incorporating the agents are considered to be embodiments of the compositions disclosed herein.

Such compositions may take any physical form, which is pharmaceutically acceptable; illustratively, they can be orally administered pharmaceutical compositions. Such pharmaceutical compositions contain an effective amount of a disclosed agent, which effective amount is related to the daily dose of the agent to be administered. Each dosage unit may contain the daily dose of a given agent or each dosage unit may contain a fraction of the daily dose, such as one-half or one-third of the dose. The amount of each agent to be contained in each dosage unit can depend, in part, on the identity of the particular agent chosen for the therapy and other factors, such as the indication for which it is given. The pharmaceutical compositions disclosed herein may be formulated so as to provide quick, sustained, or delayed release of the active ingredient after administration to the patient by employing well known procedures. The agents for use according to the methods disclosed herein may be administered as a single agent or a combination of agents.

As indicated above, pharmaceutically acceptable salts of the agents are contemplated and also may be utilized in the disclosed methods. The term “pharmaceutically acceptable salt” as used herein, refers to salts of the agents which are substantially non-toxic to living organisms. Typical pharmaceutically acceptable salts include those salts prepared by reaction of the agents as disclosed herein with a pharmaceutically acceptable mineral or organic acid or an organic or inorganic base. Such salts are known as acid addition and base addition salts. It will be appreciated by the skilled reader that most or all of the agents as disclosed herein are capable of forming salts and that the salt forms of pharmaceuticals are commonly used, often because they are more readily crystallized and purified than are the free acids or bases.

Pharmaceutically acceptable esters and amides of the agents can also be employed in the compositions and methods disclosed herein.

In addition, the methods disclosed herein may be practiced using solvate forms of the agents or salts, esters, and/or amides, thereof. Solvate forms may include ethanol solvates, hydrates, and the like.

In the present disclosure, a biomarker may be used to identify subjects that can benefit from the treatments disclosed herein. As used herein, biomarker is a measurable indicator of some biological state or condition. A biomarker is a characteristic that is objectively measured and evaluated as an indicator of normal biological processes, pathogenic processes, or pharmacologic responses to a therapeutic intervention. The biomarker may be a molecular biomarker, a cellular biomarker, a digital biomarker, a behavioral biomarker, or an imaging biomarker. The biomarker may be evaluated from a biological sample such as blood, soft tissue, tissue biopsy or sample or urine. As used herein, a biomarker may be an indicator of inflammation, an indicator of fibrosis, or a cell type specific marker.

Inflammation is mediated by the production of cytokines, chemokines, reactive oxygen species, and secondary messengers. Indicators of inflammation include cytokines and other proteins or small molecules that indicate a state of inflammation. Inflammation involves activation of the immune system in response to harmful stimuli, such as pathogens, infections, stimulants, or cellular damage. Inflammation can be classified as either acute or chronic. Generally speaking, acute inflammation is mediated by granulocytes, and chronic inflammation is mediated by mononuclear cells such as monocytes and lymphocytes.

Cytokines are a broad category of small proteins important in cell signaling and are immunomodulating agents. Cytokines include chemokines, interferons, interleukins, lymphokines, and tumor necrosis factors. An inflammatory cytokine or proinflammatory cytokine is a cytokine secreted from immune or other cells that promote inflammation. Nonlimiting examples of inflammatory cytokines include are, interleukin-1 alpha (IL-1α), IL-1 beta (IL-1β), IL-6, IL-8, IL-12, IL-17 and IL-18, tumor necrosis factor alpha (TNF-α), interferon gamma (IFNγ), granulocyte-macrophage colony stimulating factor (GM-CSF), platelet-activating factor (PAF), macrophage migration inhibitory factor (MIF) and others. Inflammatory cytokines are predominantly produced by and involved in the upregulation of inflammatory reactions. Cytokines or their receptors to which they specifically bind may be used as a biomarker including those mentioned above.

In addition to traditional biomarkers for CKD such as urinary albumin to creatinine ratio, eGFR, and the clinical parameters, age, sex, body mass index, duration of diabetes mellitus, HbA1c, history of macrovascular events, and retinopathy, other biomarkers may be used for prediction and evaluation of renal health. Biomarkers that can help determine CKD progression and associated adverse clinical end points can include groups of biomarkers that mark tubule cell injury (for example, kidney injury molecule 1, monocyte chemoattractant protein 1 (MCP-1)) and biomarkers that mark tubule cell dysfunction (e.g., al-microglobulin, uromodulin). Other biomarkers associated with fibrosis are bone morphogenetic protein-7 (BMP-7), and matrix metalloproteinase-2 (MMP-2).

Unless otherwise specified or indicated by context, the terms “a”, “an”, and “the” mean “one or more.” For example, “a molecule” should be interpreted to mean “one or more molecules.” As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus ≤10% of the particular term and “substantially” and “significantly” will mean plus or minus >10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion additional components other than the components recited in the claims. The term “consisting essentially of” should be interpreted to be partially closed and allowing the inclusion only of additional components that do not fundamentally alter the nature of the claimed subject matter.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All references, including publications, patent applications, and patents, cited herein are hereby incorporated by reference to the same extent as if each reference were individually and specifically indicated to be incorporated by reference and were set forth in its entirety herein.

Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### EXAMPLES

The following examples are illustrative and should not be interpreted to limit the scope of the claimed subject matter.

### Example 1

Wild type (WT) C57BL/6 mice (7 week old) were procured by The Jackson Laboratory and acclimatized for 1 week at standard conditions (23° C.-25° C.; 40-60% humidity with standard diet). Post acclimatization animals were subjected to unilateral ischemia reperfusion (uIRI) model of acute kidney injury (AKI) in mice as described previously. [Rajendran, G et al., 2020, J Am Soc Nephrol 31(3) 501-516] Briefly, adult C57BL/6 mice (8 weeks old) were anesthetized with xylazine (10 mg/kg intraperitoneally) and ketamine (90-120 mg/kg intraperitoneally). Post anesthesia, a small incision in the midline of the abdomen was created and the left renal pedicle was occluded with a microaneurysm clamp, while the right kidney served as an internal control. The abdomen was temporarily partly closed by covering with clean stretch film, and the body temperature was monitored with a rectal probe and maintained at 37° C. using a heating pad. After 25 minutes, the clamps were removed, and reperfusion was visually confirmed. The abdomen was closed with a 6-0 suture and the skin with Michel miniature clips. Post-ischemia reperfusion injury (IRI), mice were randomly divided into two groups: 1. Vehicle treated (WT-Veh) and 2. Syrosingopine treated (WT-Syro). Treatment with syrosingopine or vehicle was initiated on day 2 post-IRI, a time point characterized by established kidney injury. Syrosingopine (Syro) was administered intraperitoneally (7.5 mg/kg body weight, prepared in mixture of DMSO and Tween 80) on alternate days (FIG. 1A). WT-Veh group was treated with mixture of DMSO+Tween 80 (Veh) and served as control group. On days 7 and 14 post-IRI, mice were sacrificed, and kidney damage was examined using standard methods of histopathological and molecular analysis.

Histopathological Analysis Isolated kidneys were fixed in buffered formalin for 24 h and paraffin blocks were papered using automated tissue processor (Leica Biosystems). 5 μm thick paraffin kidney sections were cut and stained with hematoxylin and eosin, and Picro Sirius red. Images were captured under microscope and were analysed using Image J (National Institutes of Health [NIH]). Tubular injury was semi-quantitatively scored by determining the percentage of tubules in the corticomedullary junction that displayed necrosis, loss of brush border, cast formation, and tubular dilatation (0, unaffected; 1, 1%-25%; 2, 26%-50%; 3, 51%-75%; 4, 76%-100%). Fibrosis was assessed by the percentage of Picro-Sirius red^{+ve} area determined with ImageJ software (http://rsbweb.nih.gov/ij). For tubular injury scoring and quantification of Picro-Sirius red^{+ve} area ³10 and ³5 random visual fields of corticomedullary region, respectively were analyzed per kidney section for each sample at a magnification of ×200.

RNA and Protein Analyses RNA was isolated using Trizol from contralateral (uninjured right kidney) and IR (post unilateral clamping left kidney) kidneys and used for real-time quantitative PCR (qPCR) analysis of fibrosis and inflammatory related genes. The mouse primer sequences we used are shown in Table 1. 18S was used to normalize mRNA.

TABLE 1
Mouse primer sequences

| | | |
|---|---|---|
| Gene | Forward Primer | Reverse Primer |

Mouse

| | | |
|---|---|---|
| Loxl2 | SEQ ID NO: 1 | SEQ ID NO: 2 |
| | 5′-GATCTTCAGCCCC | 5′-CAAGGGTTGCTCTG |
| | GATGGA-3′ | GCTTGT-3′ |
| Tgfb1 | SEQ ID NO: 3 | SEQ ID NO: 3 |
| | 5′-TGGCGAGCCTTAG | 5′-TCGACATGGAGCTG |
| | TTTGGA-3′ | GTGAAA-3′ |
| Acta2 | SEQ ID NO: 5 | SEQ ID NO: 6 |
| | 5′-CCTGACGCTGAAG | 5′-TTTTCCATGTCGTC |
| | TATCCGATAG-3′ | CCAGTTG-3′ |
| Vcam1 | SEQ ID NO: 7 | SEQ ID NO: 8 |
| | 5′-TAGAGTGCAAGGA | 5′-CCGGCATATACGAG |
| | GTTCGGG-3′ | TGTGAA-3′ |
| Icam1 | SEQ ID NO: 9 | SEQ ID NO: 10 |
| | 5′-TGGATACCTGAGC | 5′-CTGCTACCTGCACT |
| | ATCACCA-3′ | TTGCC-3′ |

### Bulk RNAseq

Total RNA was extracted using TRIzol reagent (n=4). Bulk RNAseq was performed by Admera Health. RNA integrity was checked with an Agilent Technologies 2100 Bioanalyzer. Poly(A) RNA sequencing libraries were prepared. Paired-ended sequencing was performed on an Illumina sequencing system. FastQC (version v0.11.8) was applied to check the quality of raw reads. Trimmomatic (version v0.38) was applied to cut adaptors and trim low-quality bases with the default setting. STAR Aligner version 2.7.1a was used to align the reads. Picard tools (version 2.20.4) was applied to mark duplicates of mapping. The StringTie version 2.0.4 was used to assemble the RNA-Seq alignments into potential transcripts. The featureCounts (version 1.6.0)/HTSeq was used to count mapped reads for genomic features such as genes, exons, promoter, gene bodies, genomic bins and chromosomal locations. The De-Seq2 (version 1.14.1) was used to do differential analysis. Significantly (p<0.05) altered genes were used to perform hallmark enrichment analysis using Enrich® (https://maayanlab.cloud/Enrichr/) and bubble plots were prepared using R studio.

### Statistical Analysis

All data are reported as mean values±SEM. Statistical analyses were performed with Prism9.0 (GraphPad Software, La Jolla, CA) using the t test or one-way ANOVA with Sidak correction for multiple comparisons. A two-sided significance level of 5% was considered statistically significant.

### Results

Syrosingopine protects against post ischemic AKI and fibrosis. At day 14 post IRI, IR kidneys from WT-Syro group were visually better perfused as compared to WT-Veh group (FIG. 1B). Histopathological analysis showed 62% reduction in fibrosis assessed by Picro Sirius red staining at day 14 post IRI while at day 7 post IRI syrosingopine-treated mice showed 25% reduction in fibrosis (FIG. 2). Tubular injury scoring indicated reduced tubular damage in day 7 and day 14 post-IRI kidneys of syrosingopine treated mice. Furthermore, qPCR analysis showed significant reduction in the mRNA levels of profibrotic and pro-inflammatory genes lysyl oxidase-like 2 (Loxl2), transforming growth factor-beta 1 (Tgfβ1), vascular cell adhesion molecule 1 (Vcam1), and intercellular cell adhesion molecule 1 (Icam1) in IR kidneys of syrosingopine treated mice as compared to vehicle treated corresponding controls (FIG. 2). Unbiased bulk RNA-seq showed suppression of genes associated with pro-inflammatory pathways in day 7 and day 14 post-IRI kidneys of syrosingopine-treated mice compared to controls, while genes involved in metabolic pathways such as oxidative phosphorylation and fatty acid metabolism were upregulated (FIG. 3).

### Example 2

Ischemic acute kidney injury (AKI) is common in hospitalized patients and increases the risk for chronic kidney disease (CKD). Impaired endothelial cell (EC) functions are thought to contribute in AKI to CKD transition, but the underlying mechanisms remain unclear. Here, we identify a critical role for endothelial oxygen sensing prolyl hydroxylase domain (PHD) enzymes 1-3 in regulating post-ischemic kidney repair. In renal endothelium, we observed compartment-specific differences in the expression of the three PHD isoforms in both mice and humans. We found that post-ischemic concurrent inactivation of endothelial PHD1, PHD2, and PHD3 but not PHD2 alone promoted maladaptive kidney repair characterized by exacerbated tissue injury, fibrosis, and inflammation. Single-cell RNA-seq analysis of the post-ischemic endothelial PHD1, PHD2 and PHD3 deficient (PHD^{TiEC}) kidney revealed an endothelial glycolytic transcriptional signature, also observed in human kidneys with severe AKI. This metabolic program was coupled to upregulation of the SLC16A3 gene encoding the lactate exporter monocarboxylate transporter 4 (MCT4). Strikingly, treatment with the MCT4 inhibitor syrosingopine restored adaptive kidney repair in PHD^{TiEC} mice. Mechanistically, MCT4 inhibition suppressed pro-inflammatory EC activation reducing monocyte-endothelial cell interaction. Our findings suggest halting AKI to CKD transition based on selectively targeting the endothelial hypoxia-driven glycolysis/MCT4 axis.

Ischemia is the most common cause of acute kidney injury (AKI), a condition that affects approximately 10-15% of all hospitalized patients, and more than 50% of critically ill patients. Failure to fully recover from AKI may lead to persistent functional impairment and development of chronic and progressive kidney disease, leading to end stage renal disease (ESRD). Given the lack of treatments that successfully promote kidney repair, it is crucial to better understand the mechanisms that regulate AKI to chronic kidney disease (CKD) transition and identify novel therapeutic targets.

Severe or prolonged ischemic injury can overwhelm the kidney's reparative ability and trigger a cascade of detrimental responses within the renal microenvironment. Tubular epithelial cells undergo dedifferentiation, apoptosis, and impaired regeneration, leading to the disruption of the nephron structure. Immune cells, such as neutrophils and macrophages, infiltrate the injured tissue and perpetuate parenchymal cell injury and fibrosis. Fibroblasts become hyperactive, excessively depositing collagen and promoting tissue scarring. In this milieu, endothelial cells (ECs) form a metabolically dynamic barrier that when impaired promotes immune cell migration, dysregulated vascular tone, and permeability. As a result, the reduced blood flow limits oxygen delivery to affected areas leading to hypoxia. Besides the microvasculature dysfunction, the induction of hypoxia in the post-ischemic kidney involves multiple factors such as increased oxygen consumption, mitochondrial dysfunction, reduced oxygen carrying capacity due to anemia, and hindered oxygen diffusion by extracellular matrix (ECM) buildup. While hypoxia has been linked to CKD progression, the underlying cellular mechanisms remain poorly understood.

Being in direct contact with the blood, ECs are well equipped to detect changes in oxygen levels through prolyl-4-hydroxylase domain-containing proteins (PHDs, also known as EGLNs), which control the stability of HIF-α. In the presence of oxygen, PHDs hydroxylate HIF-α subunits at highly conserved proline residues promoting their ubiquitination by the VHL (von-Hippel-Lindau) enzyme and ultimate proteasomal degradation. Under hypoxia, the reduced catalytic activity of PHDs results in HIF-α stabilization. HIF-α then translocates to the nucleus, where it dimerizes with the constitutively expressed HIF-β subunit (also known as ARNT), regulating the transcription of a broad array of genes. Genes involved in survival, metabolism, and angiogenic activity of vascular ECs are regulated by HIF with major implications in controlling vascular development and disease settings such as cancer and inflammatory processes. While PHD2 is considered the main oxygen sensor regulating HIF-α protein levels in normoxia, other PHD isoforms contribute to HIF regulation in particular cell types or conditions, dependent on their abundance. In vascular endothelium, significant heterogeneity across organs introduces an additional layer of complexity. For instance, we and others have shown a crucial role for PHD2 in regulating endothelial HIF-α in the lung, and endothelial PHD2 inactivation led to pulmonary hypertension. On the other hand, our recent studies showed decreased responsiveness of the kidney endothelial HIF-α to PHD2 loss alone [Rajendran G, Schonfeld M P, Tiwari R, Huang S, Torosyan R, Fields T, et al. Inhibition of Endothelial PHD2 Suppresses Post-Ischemic Kidney Inflammation through Hypoxia-Inducible Factor-1. J Am Soc Nephrol. 2020; 31(3):501-16]. Indeed, the expression of other PHD isoforms in the renal vascular bed as demonstrated by single-cell RNA sequencing (scRNA-seq) data implied their potential contribution in regulating endothelial HIF activation in the kidney. Due to this complexity of the PHD/HIF system in vascular endothelium, their relevance in kidney repair after ischemic injury remain elusive.

To delineate the role of endothelial oxygen sensing in regulating post-ischemic kidney repair, we have generated a set of conditional mouse strains in which we induced endothelial inactivation of PHD2 alone, or in combination with PHD1 and PHD3 taking advantage of the Cdh5Cre(PAC)ER transgenic mice [Rajendran, G (supra); Sorensen I, Adams RH, and Gossler A. DLL1-mediated Notch activation regulates endothelial identity in mouse fetal arteries. Blood. 2009; 113(22):5680-8]. While PHD2 inactivation alone did not alter kidney repair, simultaneous post-ischemic inactivation of endothelial PHD1, PHD2 and PHD3 induced robust HIF activation and exacerbated fibrosis, inflammation, and capillary dropout. ScRNA-seq analysis revealed a signature significant for endothelial hypoxia and glycolysis in association with proinflammatory responses. Finally, using a pharmacologic approach, we identified the hypoxia-regulated lactate exporter monocarboxylate transporter 4 (MCT4) as a target to suppress maladaptive pro-inflammatory responses and halt AKI to CKD transition.

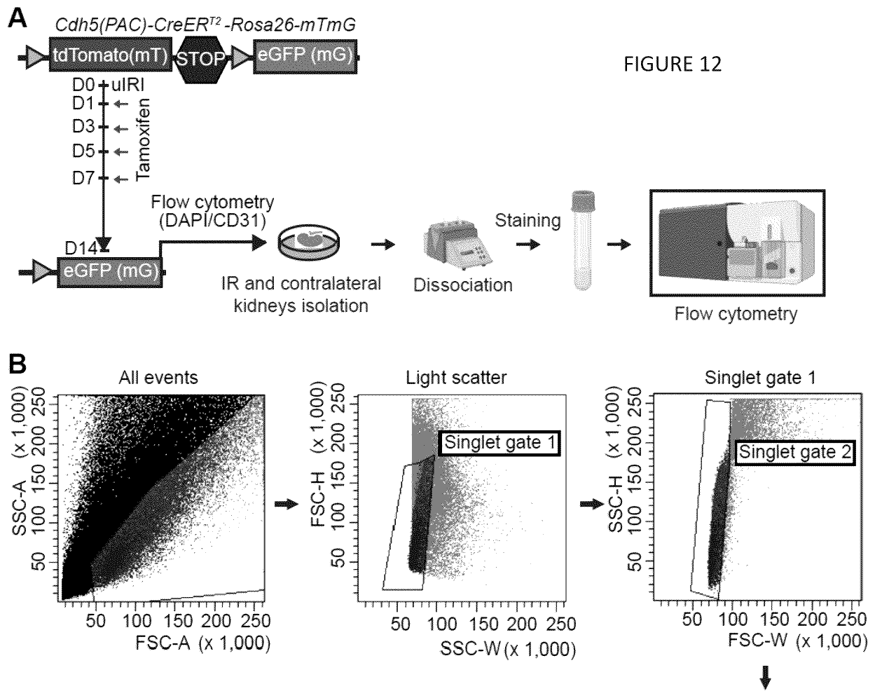

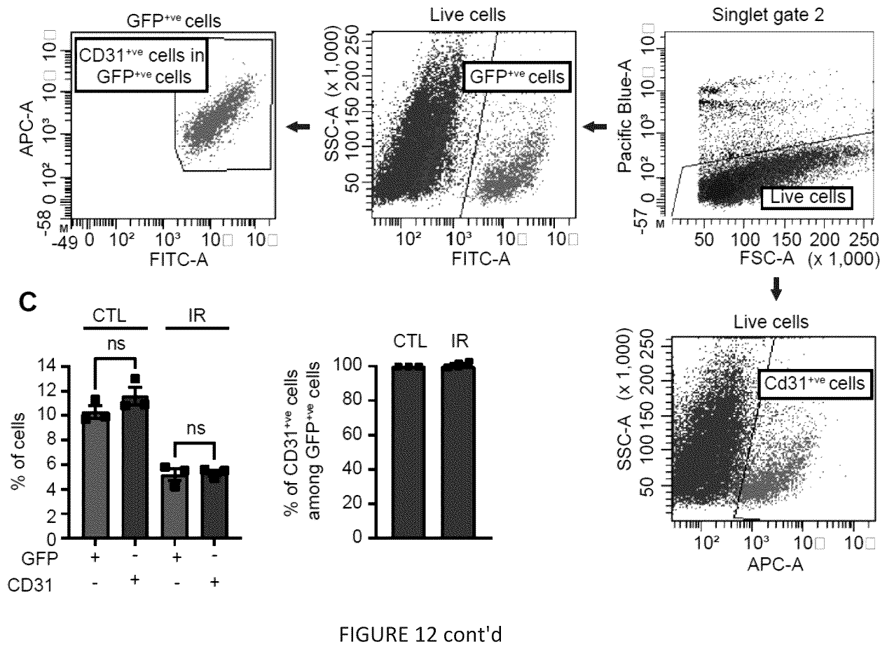

Post-ischemic inactivation of endothelial PHD2 does not affect post-ischemic kidney injury Constitutive or acute inactivation of PHD2 in ECs prior to kidney ischemia-reperfusion injury (IRI) prevents post-ischemic injury and inflammation. While these findings show the beneficial effects of endothelial PHD/HIF signaling to promote kidney resilience against ischemic insult, they cannot be extrapolated to kidney repair following established injury, in which distinct responses control regeneration of the damaged tissue. To ask whether inhibition of endothelial PHD2 regulates kidney repair, we took advantage of tamoxifen-inducible Cdh5-CreER^{T2};Phd2^{f/f} mice (PHD2^{iEC}) [Rajendran, G., et al. (supra), Sorenson, I. et al, (supra)], which allow induction of recombination following established kidney injury. We first assessed the endothelial recombination efficiency achieved when tamoxifen treatment started on day 1 post renal unilateral IRI (uIRI) and included 4 total doses given every other day, as indicated in FIG. 12A. Using Cdh5(PAC)CreER^{T2}-Rosa26-mTmG mice, which allow tracking of recombinant cells by GFP expression, we performed flow cytometry (FIG. 12B) and found that ˜100% of GFP^{+ve} cells in day 14 post-ischemic kidneys were also CD31^{+ve}. This recombination efficiency was comparable to the contralateral kidneys. Otherwise, at day 14 post-IRI, there was a ˜50% reduction in kidney ECs, which is expected in the setting of IRI-induced capillary rarefaction (FIG. 12C).

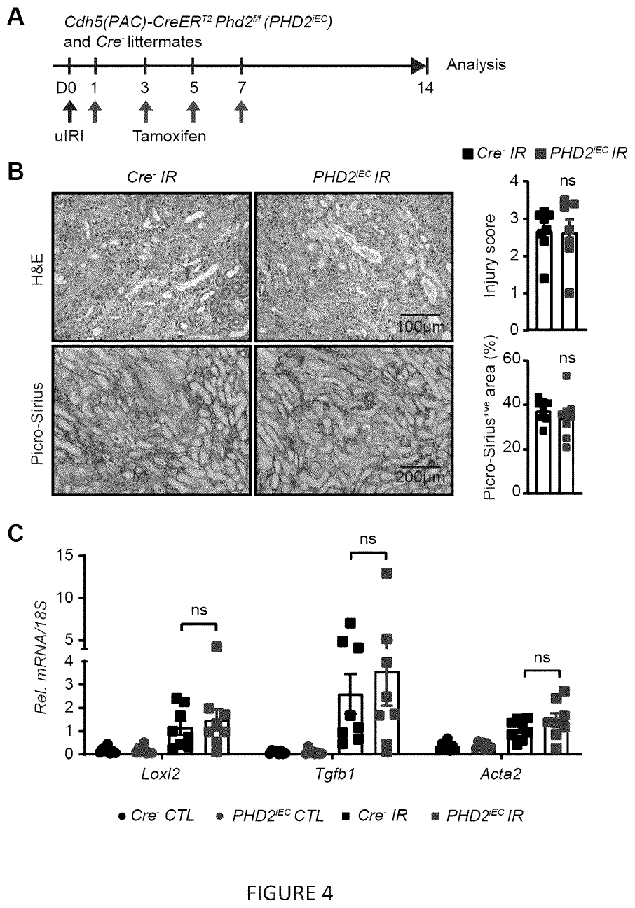

After validating the recombination efficiency of the Cdh5(PAC)CreER^{T2} system in the post-ischemic kidney, adult male PHD2^{iEC} mice and their Cre⁻ littermates were subjected to uIRI, followed by four tamoxifen injections and analysis on day 14, as indicated in FIG. 4A. Assessment of tubular injury on H&E-stained sections of day 14 post-ischemic kidneys revealed that PHD2^{iEC} had comparable tubular damage to Cre⁻ littermate controls. Furthermore, quantitative analysis of Picro-Sirius red staining showed no significant difference in collagen accumulation between mutants and controls (FIG. 4B). Finally, day 14 post-ischemic kidneys showed significantly increased transcripts of profibrotic genes lysyl oxidase-like 2 (Loxl2), transforming growth factor beta 1 (Tgfb1), and smooth muscle cell a-actin (Acta2) compared to their corresponding contralateral, but there was no significant change between the two genotypes (FIG. 4C). Therefore, acute post-ischemic endothelial inactivation of PHD2 does not affect kidney repair and transition to CKD following IRI.

### Kidney Endothelium Shows Compartment-Specific Differential Expression of PHD1, 2, and 3 in Mice and Humans

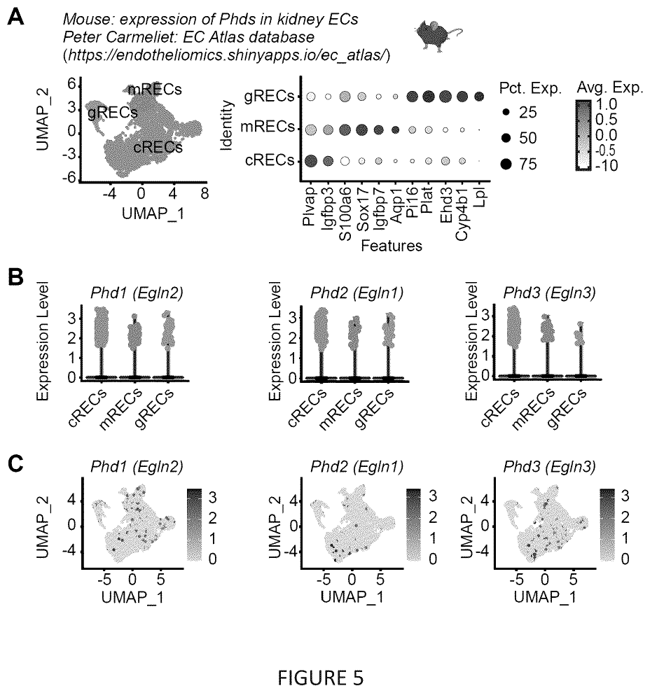

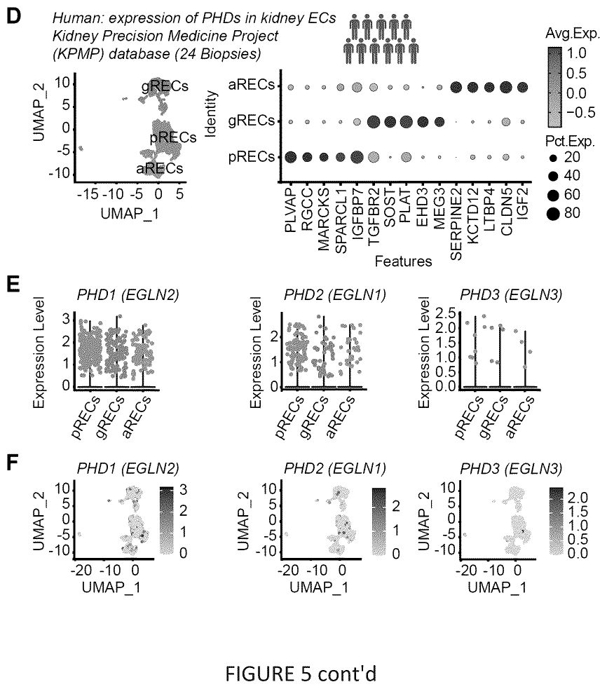

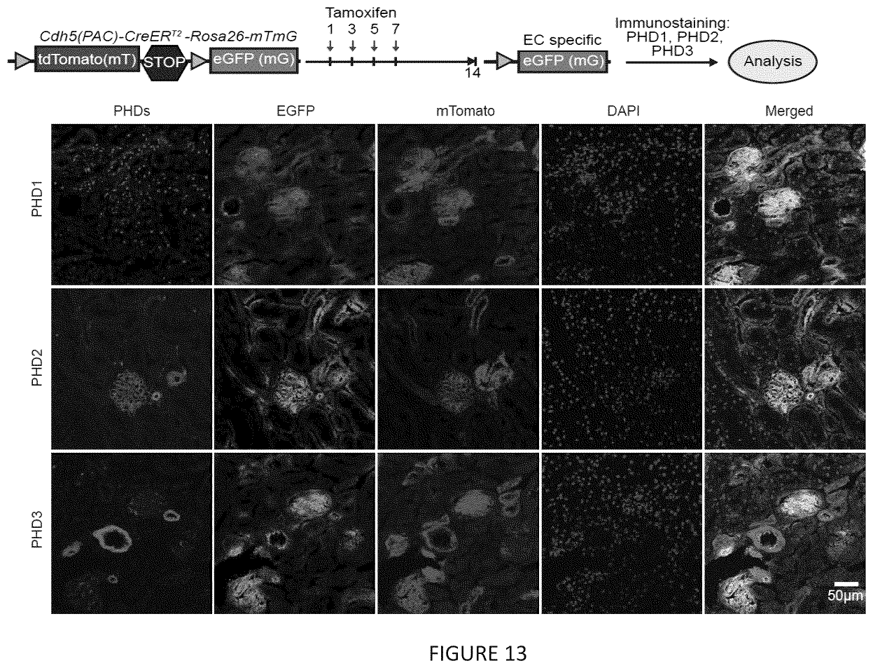

Inactivation of endothelial PHD2 alone was not sufficient to stabilize HIF in the renal endothelium, raising the possibility for significant contributions of PHD1 and/or PHD3 in regulating renal endothelial HIF protein levels. Indeed, while PHD2 is considered the primary regulator of HIF-α stability under normal conditions, several studies have suggested a significant role for other PHD isoforms in controlling HIF-α activity under different circumstances depending on differential induction and cell type specific PHD expression. Nevertheless, the function of PHD isoforms in different vascular beds remains unexplored. To better understand the role of oxygen sensors in renal vasculature, the inventors took advantage of publicly available scRNA-seq data and characterized the expression of different Phd isoforms in kidney EC. We first analyzed the expression of Phd1 (Egln2), Phd2 (Egln1), and Phd3 (Egln3) in scRNA-seq data of renal ECs (RECs) extracted from the murine EC Atlas database (FIG. 5A-C) [Kalucka J, de Rooij L, Goveia J, Rohlenova K, Dumas S J, Meta E, et al. Single-Cell Transcriptome Atlas of Murine Endothelial Cells. Cell. 2020; 180(4):764-79 e20.]. We found that Phd1 was ubiquitously expressed, whereas Phd2 and Phd3 were mainly expressed in cortical and medullary RECs. Glomerular RECs showed particularly low expression for Phd3. To further validate the differential expression of PHDs in murine kidney endothelium, we immunostained kidney sections from tamoxifen treated Cdh5(PAC)CreER^{T2}-Rosa26-mTmG reporter mice with antibodies against PHD1, PHD2, and PHD3. Immunolabelling confirmed ubiquitous expression of PHD1, expression of PHD2 in cortical and medullary RECs, and low expression of PHD3 in glomerular RECs (FIG. 13).

The inventors sought to characterize the expression of PHD isoforms within human kidney endothelium. To this end, the inventors analyzed scRNA-seq data of human kidneys available through KPMP (FIG. 5D-F and Table 2) [Menon R, Otto EA, Hoover P, Eddy S, Mariani L, Godfrey B, et al. Single cell transcriptomics identifies focal segmental glomerulosclerosis remission endothelial biomarker. JCI Insight. 2020;5(6)]. ECs were identified by the expression of CD34 and endomucin (EMCN) and were clustered to three sub-clusters based on the expression of previously validated specific markers; PLVAP in peritubular ECs, EHD3 in glomerular and SERPINE2 in arteriolar ECs (FIG. 5D)[Menon, R., et al., (supra)]. This analysis demonstrated that PHD1 was the most highly expressed isoform whereas PHD3 showed the lowest expression. Among the different compartments, PHD2 was predominantly expressed in peritubular capillary ECs (FIG. 5E and F). Together these studies suggest endothelial compartment-specific differences in the expression of the three PHD isoforms in mice and humans.

TABLE 2
List of GEO numbers and details of samples which were
used to assess the level of Eglns in human kidney ECs.

| | | | | Subject | |
|---|---|---|---|---|---|
| Samples | GEO | Source name | Organism | status | Tissue |
| 1 | GSM4191941 | Tumor- | Homo | Normal/ | Adult |
| | | nephrectory | sapiens | healthy | kidney |
| 2 | GSM4191942 | Tumor- | Homo | Normal/ | Adult |
| | | nephrectory | sapiens | healthy | kidney |
| 3 | GSM4191943 | Tumor- | Homo | Normal/ | Adult |
| | | nephrectory | sapiens | healthy | kidney |
| 4 | GSM4191944 | Tumor- | Homo | Normal/ | Adult |
| | | nephrectory | sapiens | healthy | kidney |
| 5 | GSM4191945 | Tumor- | Homo | Normal/ | Adult |
| | | nephrectory | sapiens | healthy | kidney |
| 6 | GSM4191946 | Tumor- | Homo | Normal/ | Adult |
| | | nephrectory | sapiens | healthy | kidney |
| 7 | GSM4191947 | Tumor- | Homo | Normal/ | Adult |
| | | nephrectory | sapiens | healthy | kidney |
| 8 | GSM4191948 | Tumor- | Homo | Normal/ | Adult |
| | | nephrectory | sapiens | healthy | kidney |
| 9 | GSM4191949 | Tumor- | Homo | Normal/ | Adult |
| | | nephrectory | sapiens | healthy | kidney |
| 10 | GSM4191950 | Tumor- | Homo | Normal/ | Adult |
| | | nephrectory | sapiens | healthy | kidney |
| 11 | GSM4191951 | Tumor- | Homo | Normal/ | Adult |
| | | nephrectory | sapiens | healthy | kidney |
| 12 | GSM4191952 | Living donor | Homo | Normal/ | Adult |
| | | | sapiens | healthy | kidney |
| 13 | GSM4191953 | Living donor | Homo | Normal/ | Adult |
| | | | sapiens | healthy | kidney |
| 14 | GSM4191954 | Living donor | Homo | Normal/ | Adult |
| | | | sapiens | healthy | kidney |
| 15 | GSM4191955 | Tumor- | Homo | Normal/ | Adult |
| | | nephrectory | sapiens | healthy | kidney |
| 16 | GSM4191956 | Tumor- | Homo | Normal/ | Adult |
| | | nephrectory | sapiens | healthy | kidney |
| 17 | GSM4191957 | Tumor- | Homo | Normal/ | Adult |
| | | nephrectory | sapiens | healthy | kidney |
| 18 | GSM4191958 | Tumor- | Homo | Normal/ | Adult |
| | | nephrectory | sapiens | healthy | kidney |
| 19 | GSM4191959 | Tumor- | Homo | Normal/ | Adult |
| | | nephrectory | sapiens | healthy | kidney |
| 20 | GSM4191960 | Surveillance | Homo | Normal/ | Adult |
| | | biopsy | sapiens | healthy | kidney |
| 21 | GSM4191961 | Surveillance | Homo | Normal/ | Adult |
| | | biopsy | sapiens | healthy | kidney |
| 22 | GSM4191962 | Surveillance | Homo | Normal/ | Adult |
| | | biopsy | sapiens | healthy | kidney |
| 23 | GSM4191963 | Surveillance | Homo | Normal/ | Adult |
| | | biopsy | sapiens | healthy | kidney |
| 24 | GSM4191964 | Surveillance | Homo | Normal/ | Adult |
| | | biopsy | sapiens | healthy | kidney |

### Post-Ischemic Concurrent Inactivation of Endothelial PHD1, 2 and 3 Promotes HIF Activation and Maladaptive Kidney Repair

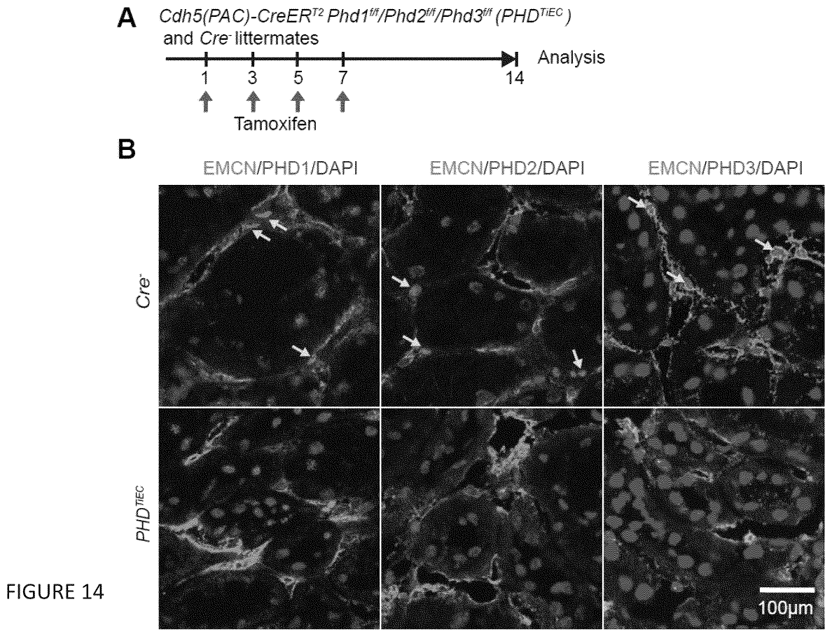

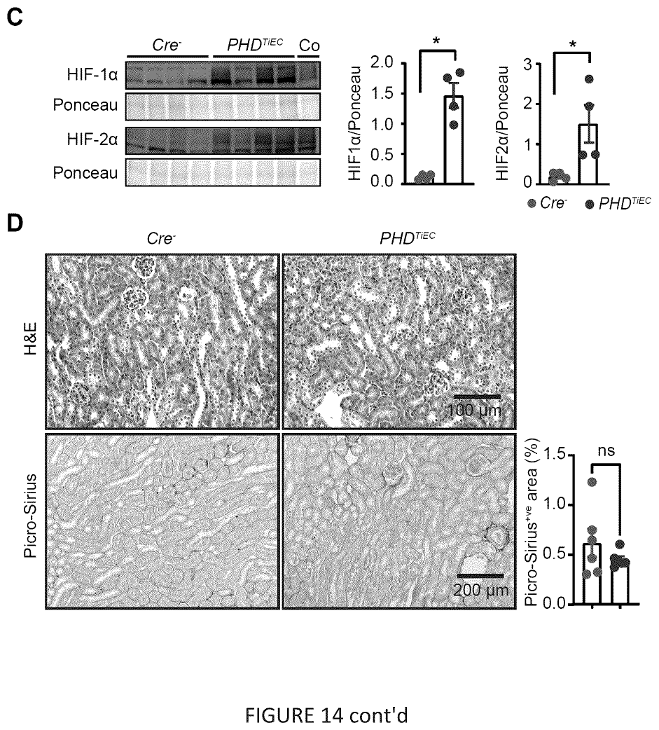

Because we found that all PHD isoforms are expressed in kidney endothelium with variable abundance within different compartments, we next generated mice that allow acute endothelial specific inactivation of PHD1, PHD2 and PHD3 (Cdh5(PAC)CreER^{T2}; Phd1^{f/f}Phd2^{f/f}Phd3^{f/f} mice; PHD^{TiEC}) and predicted that the resulting mutants will show HIF stabilization in endothelium. One week after the completion of tamoxifen treatment (FIG. 14A), co-immuno staining with antibodies against PHD1, 2 and 3 and EMCN demonstrated successful ablation of PHD1, 2 and 3 in kidney ECs of PHDT^{TiEC} compared to Cre⁻ littermates (FIG. 14B). Further, immunoblot assessment of kidney nuclear extracts isolated from PHD^{TiEC} and Cre⁻ littermates revealed significant stabilization of HIF-1α and HIF-2α in the kidneys of PHD^{TiEC} mice (FIG. 14C). Baseline histopathological analysis showed normal kidney morphology without fibrosis in PHD^{TiEC} mice as indicated by H&E and Picro-Sirius red staining (FIG. 14D).

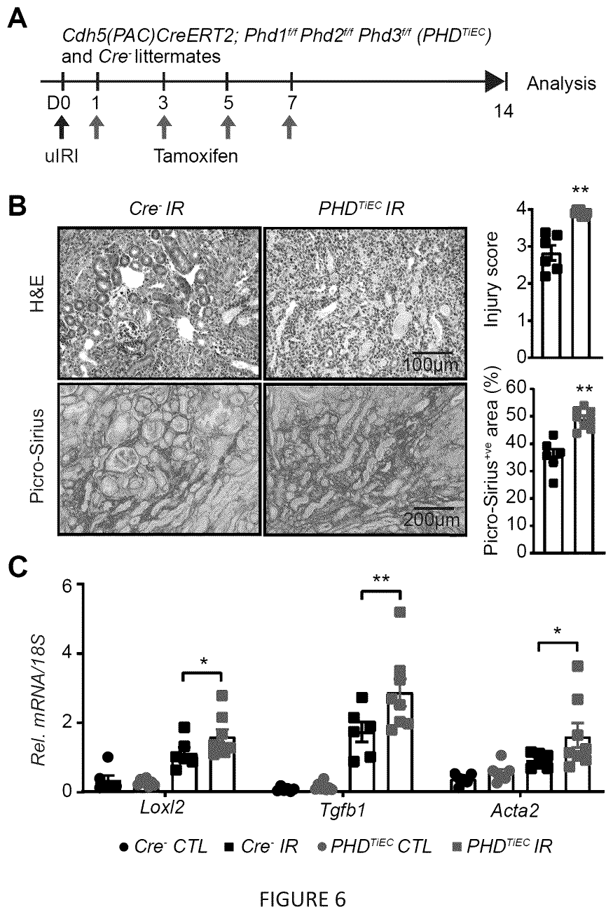

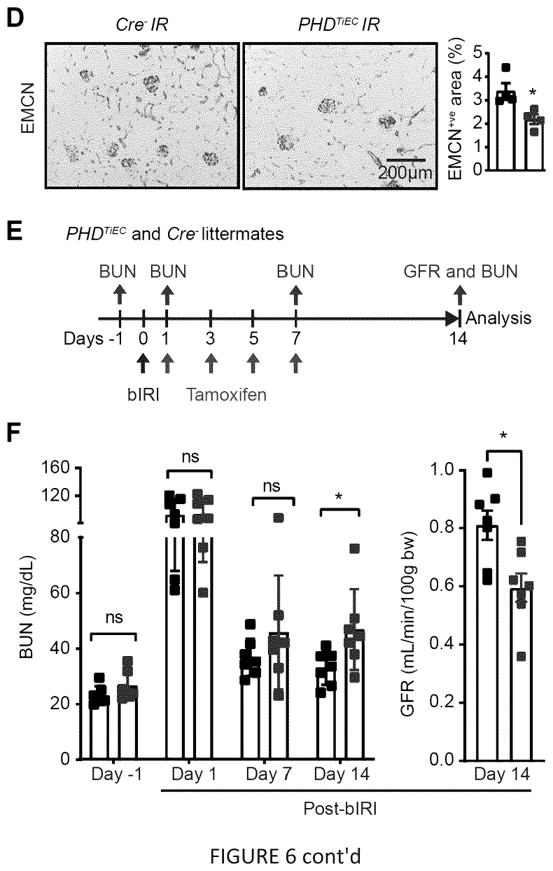

To determine the impact of post-ischemic inactivation of endothelial PHD1, PHD2 and PHD3 on kidney repair following IRI, Cre⁻ and PHD^{TiEC} adult mice were subjected to unilateral renal artery clamping for 25 minutes followed by tamoxifen treatment and analysis on day 14 (FIG. 6A). Notably, histological analysis of day 14 post-ischemic kidneys revealed higher tubular dilatation and cast formation in PHD^{TiEC} mice than in Cre⁻ controls (FIG. 6B). Furthermore, day 14 post-ischemic kidneys from PHD^{TiEC} mutants showed ˜38% increase in collagen deposition compared to Cre⁻ littermates as indicated by Picro-Sirius red staining (n=6-8, P<0.05) (FIG. 6B). Accordingly, qPCR analysis also showed significant transcriptional induction of pro-fibrotic genes Loxl2 (P<0.05), Tgfb1 (P<0.01), and Acta2 (P<0.05) indicating increased post-ischemic kidney fibrosis in PHD^{TiEC} mutants compared to Cre controls (FIG. 6C). Because peritubular capillary rarefaction is a hallmark of fibrotic kidneys, we also assessed capillary density by EMCN staining and found that EMCN^{+ve} area was reduced by 35% in day 14 post-ischemic kidneys from PHD^{TiEC} mice (n=4, P<0.05) (FIG. 6D).

To assess the impact of post-ischemic endothelial inactivation of Phd1, 2, and 3 on recovery of kidney function after IRI, PHD^{TiEC} and Cre⁻ littermate mice were subjected to bilateral renal IRI (bIRI), followed by tamoxifen treatment (FIG. 6E). Blood urea nitrogen (BUN) levels were measured one day prior to surgery (baseline) and on days 1, 7 and 14 after bIRI surgery. As expected, on day 1 after bIRI, BUN levels increased to similar degree for both genotypes without a significant difference on day 7. However, on day 14 post bIRI, PHD^{TiEC} had significantly increased BUN levels by 43% compared to Cre⁻ mice (P<0.05). In accordance, GFR measurements on day 14 post bIRI showed 26% reduction in GFR of PHD^{TiEC} mice compared to their Cre-littermates (n=7, P<0.01) (FIG. 6F). In conclusion, our findings demonstrate that EC-specific inactivation of PHD1, PHD2 and PHD3 after IRI promotes maladaptive kidney repair characterized by tubular injury, interstitial fibrosis and capillary drop-out; these changes lead to impaired recovery of kidney function.

scRNA-Seq Reveals Distinct Cell-Specific Responses in Post-Ischemic PHD^{TiEC} Mutant Kidney

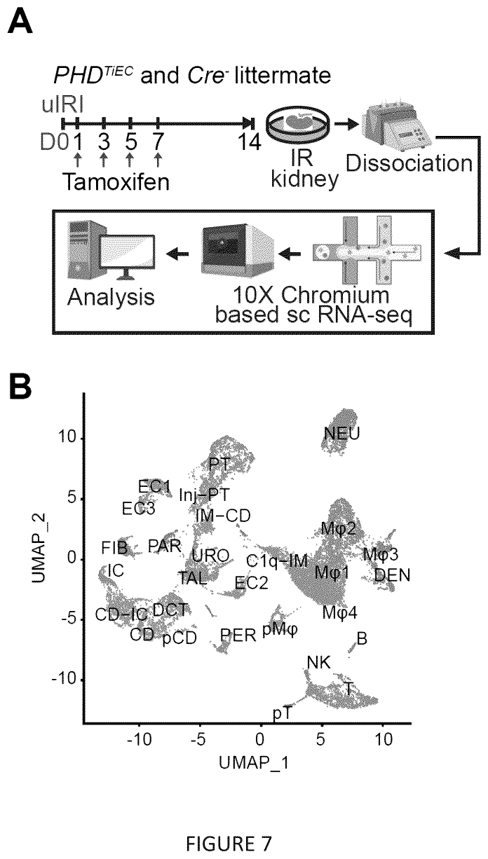

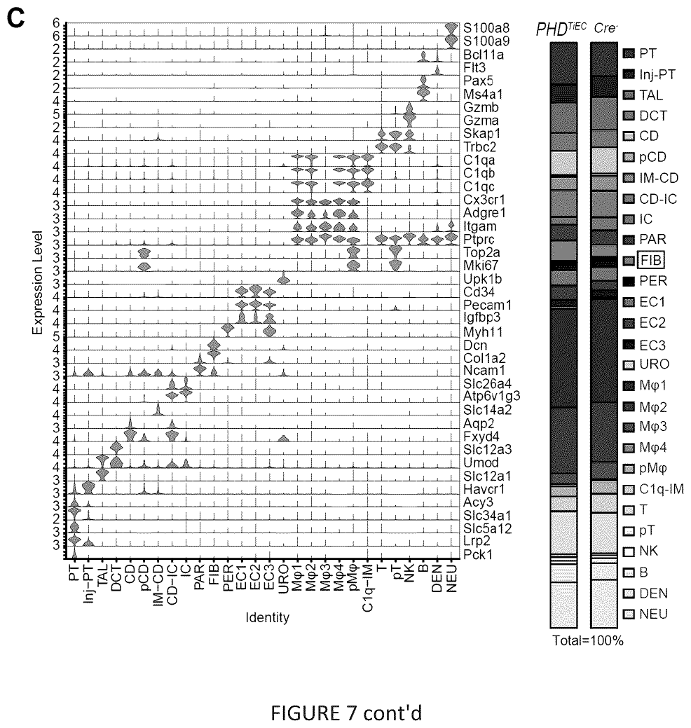

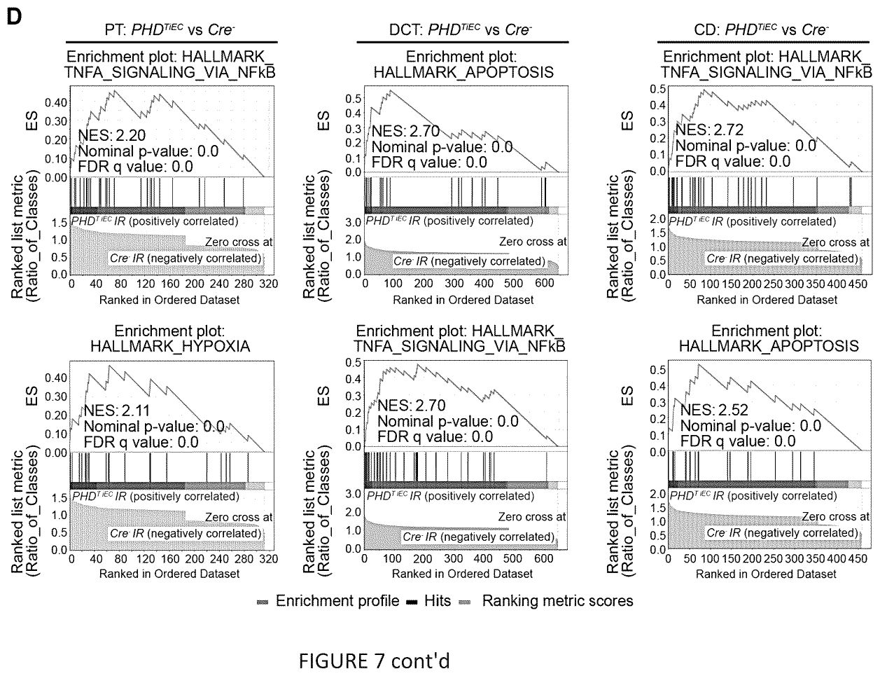

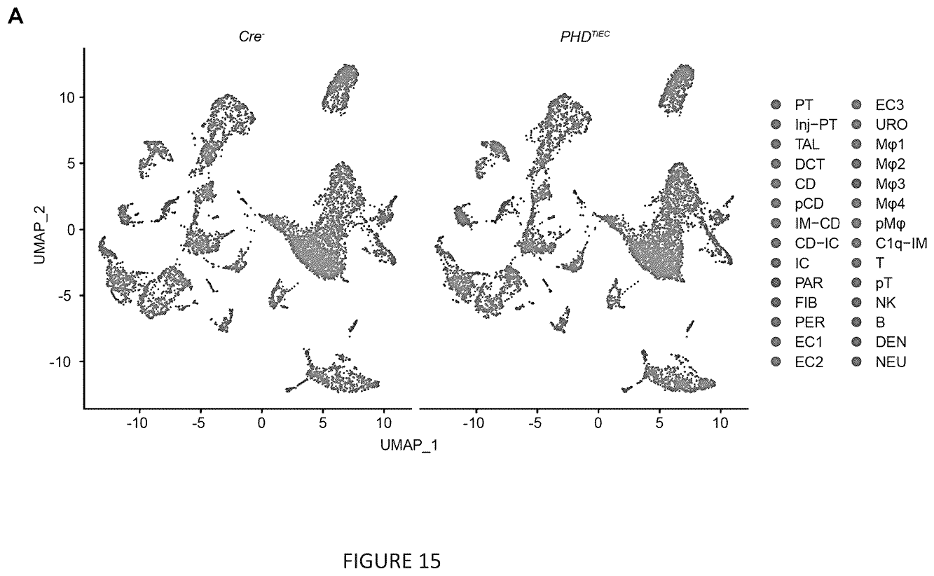

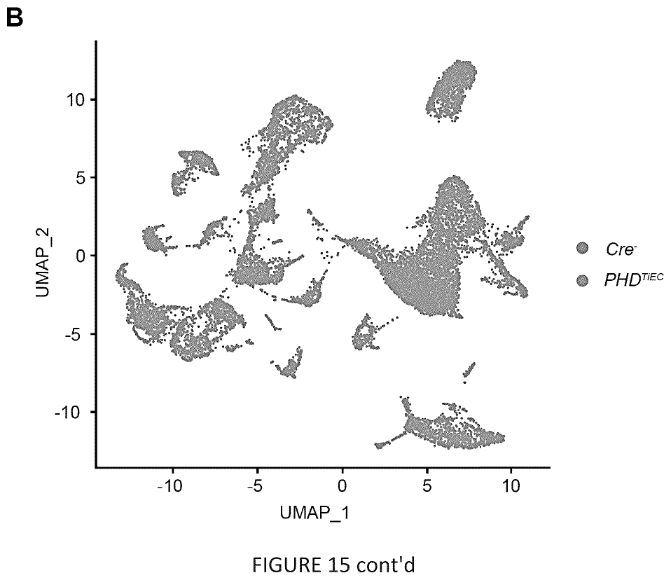

To investigate the impact of post-ischemic endothelial PHD inactivation on cell-type specific alterations in the context of kidney repair following IRI, we carried out scRNA-seq on day 14 post-ischemic kidneys from PHD^{TiEC} and Cre⁻ control mice. Using the 10× Genomics Chromium platform, we aimed for 10,000 cells per group (FIG. 7A). After quality filtering, a total of 16,698 (7,955 from Cre⁻ and 8,743 from PHD^{TiEC}) cells remained for subsequent analysis. We identified 28 different cell clusters using unsupervised graph-based clustering followed by principal component analysis (PCA) and uniform manifold approximation and projection (UMAP) (FIG. 7B). When we overlayed Cre⁻ IR and PHD^{TiEC} IR samples, we observed similar cell clusters in each genotype (FIG. 15). Based on expression of reported canonical marker genes (Table 3), we identified all expected kidney cell types; proximal tubule (PT), Injured PT, thick ascending limb (TAL), distal convoluted tubule (DCT), collecting duct (CD), proliferating CD (pCD), inner medullary collecting duct (IM-CD), collecting duct-intercalated cells (CD-IC), intercalated cells (IC), parietal cells (PAR), fibroblasts (FIB), pericytes (PER), endothelial cells 1-3 (EC1-3), urothelial cells (URO), Macrophages 1-4 (Mφ1-4), proliferating macrophages (pMφ), C1q (C1qa, C1qb and C1qc) expressing immune cells (C1q-IM), T cells (T), proliferating T cells (pT), natural killer cells (NK), B cells (B), dendritic cells (DEN), and neutrophils (NEU) (FIG. 7C). Consistent with the aforementioned increased fibrosis, analysis of the relative cell proportions for each genotype showed increased fibroblasts by ˜1.9-fold in the post-ischemic PHD^{TiEC} kidney compared to control. Next, we performed differential gene expression analysis for tubular clusters between the two genotypes. After applying cutoff criteria of a log₂ fold change >0.2 and adjusted P<0.05, PT, Inj-PT, TAL, DCT, CD, and CD-IC clusters of PHD^{TiEC} IR kidney showed 163, 90, 209, 269, 211 and 250 differentially expressed genes (DEGs), respectively. Gene set enrichment analysis (GSEA) demonstrated significant differences in tubular cells between PHD^{TiEC} and Cre-Within the tubular clusters, the Top Hallmark gene signatures were TNFA signalling via NFkB and Hypoxia pathways for PT; Apoptosis, TNFA signalling via NFkB, and Hypoxia pathway for DCT; TNFA signalling via NFkB, Apoptosis, and Hypoxia pathway for CD (FIG. 7D). Overall, these results show that the exacerbated fibrotic response in post-ischemic kidneys of PHD^{TiEC} mutants is associated with a pro-inflammatory signature in tubular cells.

TABLE 3
List of used marker genes to identify different cell cluster of
scRNA seq data of Cre⁻ and PHD^{TiEC} mice and their references.

| Marker genes | Cell cluster | Reference |
|---|---|---|
| Pck1, Lrp2, Slc5a12, | Proximal tubule (PT) | (1, 2) |
| Slc34a1 | | |
| Havcr1 | Injured PT (Inj-PT) | (1) |
| Umod, Slc12a1 | Thick ascending limb (TAL) | (1) |
| Slc12a3 | Distal convoluted tubule (DCT) | (3) |
| Fxyd4, Aqp2 | Collecting duct (CD) | (4) |
| Slc14a2 | Inner medullary collecting duct | (5) |
| | (IM-CD) | |
| Atp6v1g3, Scl26a4 | Intercalated cells (IC) | (1, 6) |
| Ncam1 | Parietal cells (PAR) | (7) |
| Col1a2, Col1a2, Dcn | Fibroblasts (FIB) | (2, 8, 9) |
| Myhl1, Acta2, | Pericytes (PER)/smooth muscle | (9) |
| | cells | |
| Cd34, Pecam1, Igfbp3 | Endothelial cells (EC) | (1, 10) |
| Upk1b | Urothelial cells (URO) | (11) |
| Mki67, Top2a | Proliferating cells (p) | (1) (12) |
| Ptprc | Immune cells | (6) |
| Itgam | Myeloid cells | (12) |
| Adgre1, Cx3cr1 | Macrophages (Mj) | (1, 12) |
| C1qa, C1qb, C1qc | C1qa, C1qb and C1qc expressing | (13, 14) |
| | immune cell (C1q-IM); potentially | |
| | resident macrophage/monocyte | |
| Trbc2, Skap1 | T cells | (14, 15) |
| GZMA, GZMB | Natural killer cells (NK cells) | (16) |
| Ms4a1, Pax5, Bcl11a | B cells | (15, 17-19) |
| Flt3 | Dendritic cells (DC) | (20, 21) |
| S100a8, S100a9 | Neutrophils (NEU) | (22) |

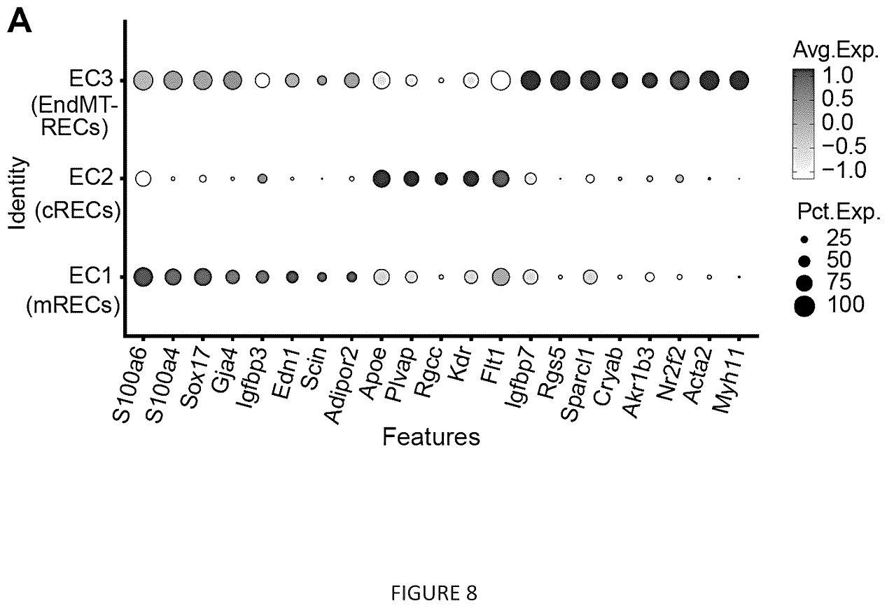

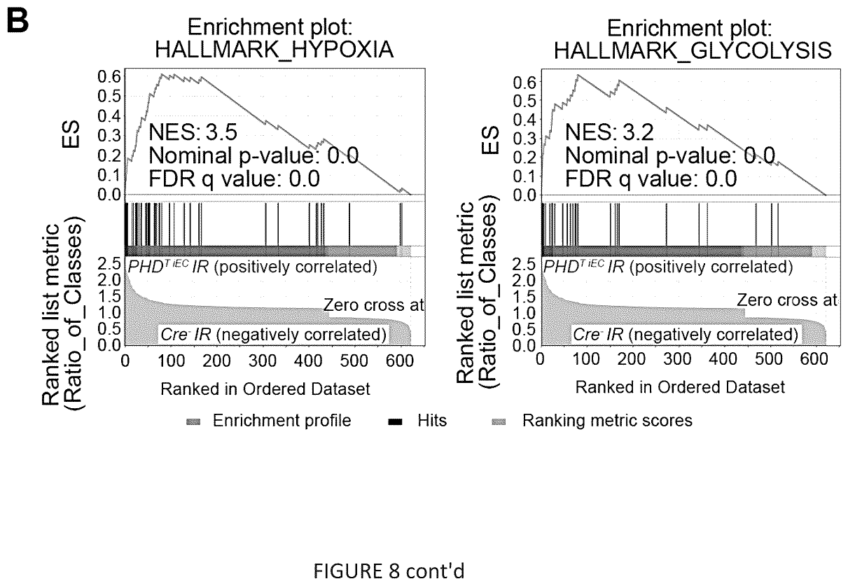

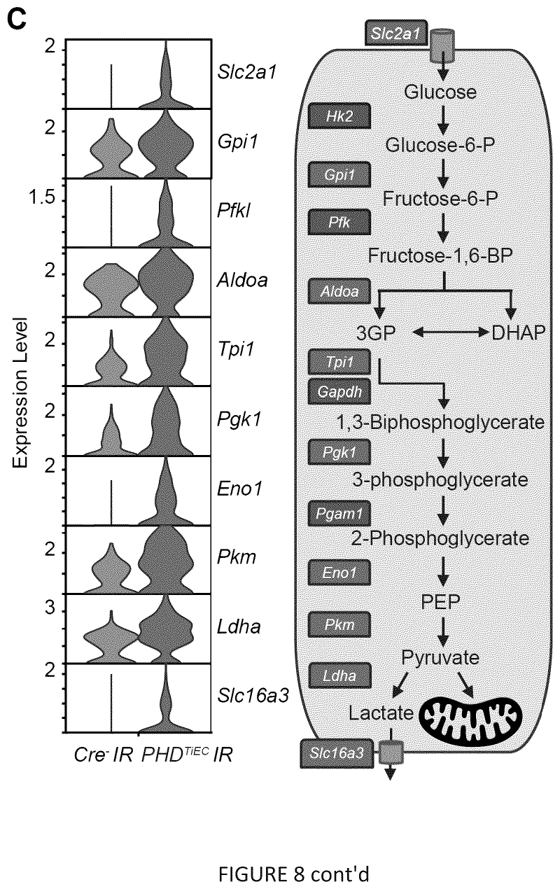

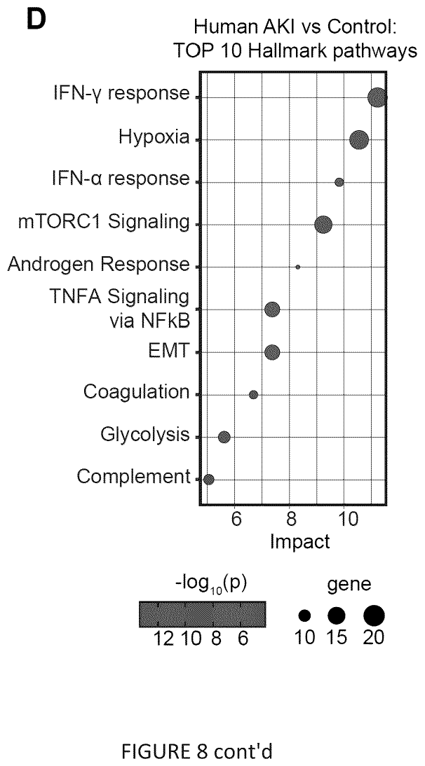

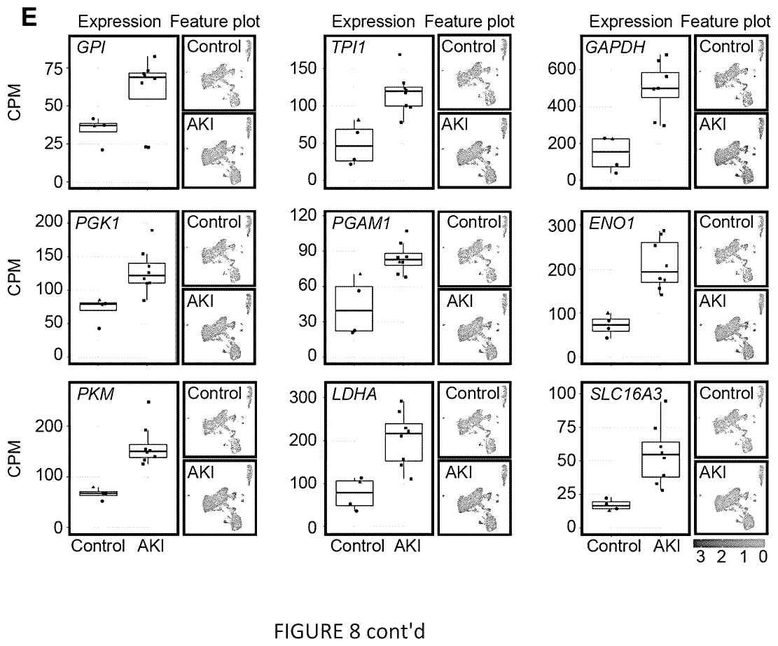

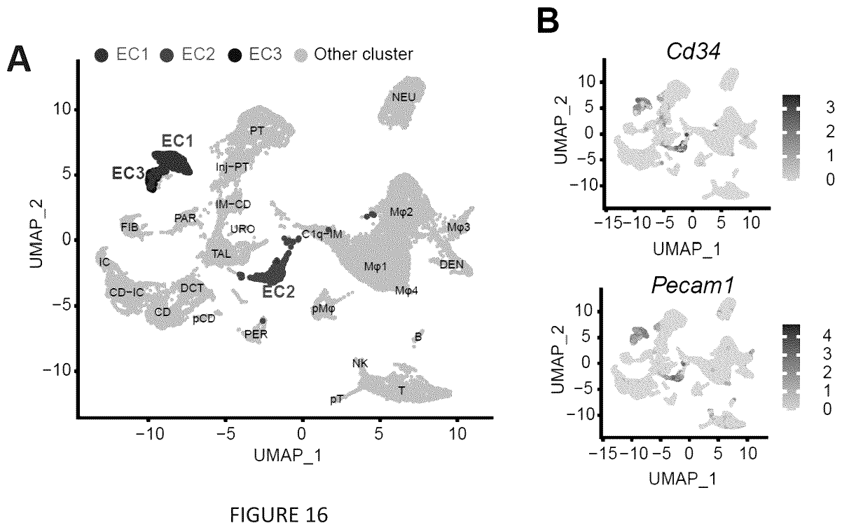

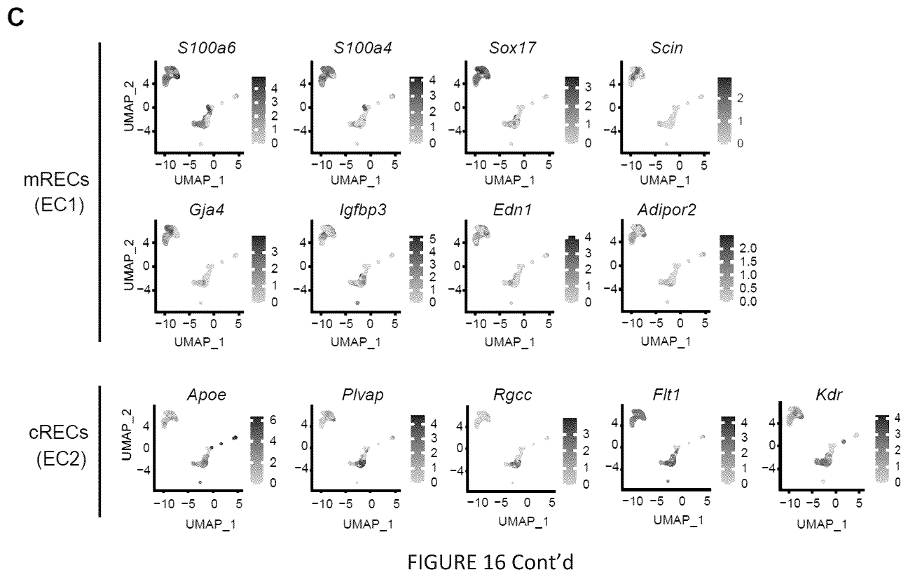

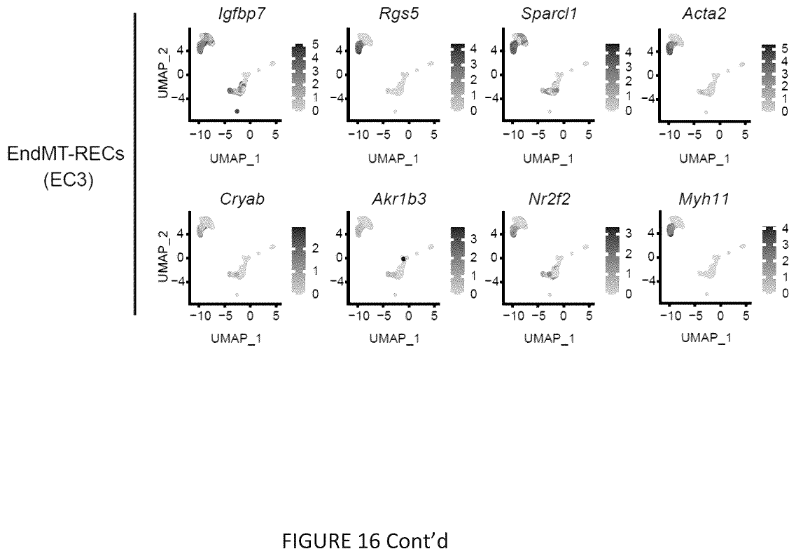

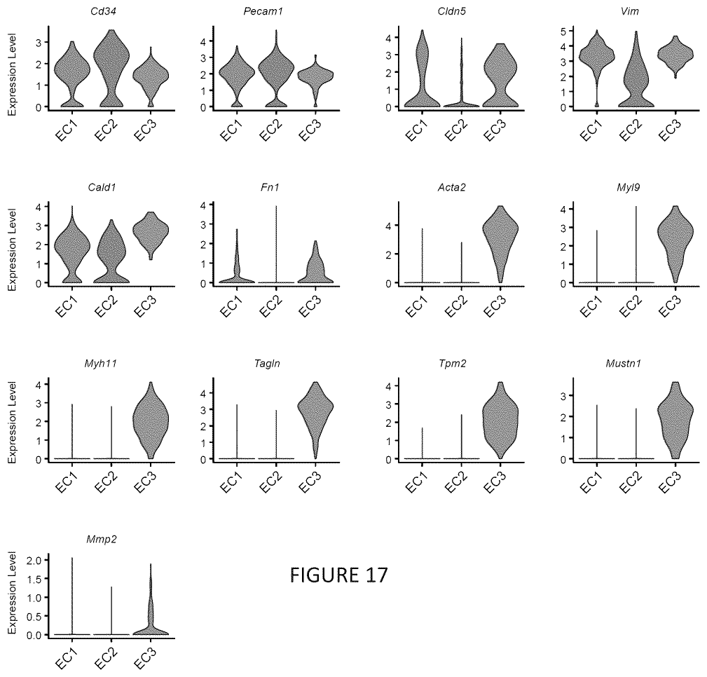

Medullary renal ECs of post-ischemic PHD^{T}1E^{C} mutant kidney show increased glycolysis Next, we focused on the three EC clusters annotated as medullary RECs (mRECs), cortical Renal ECs (cRECs), and RECs expressing mesenchymal markers (EndMT-RECs) based on marker genes established from the Carmneliet group [Dumas S J, et al. Single-Cell RNA Sequencing Reveals Renal Endothelium Heterogeneity and Metabolic Adaptation to Water Deprivation. J Am Soc Nephrol. 2020; 31(1):118-38] and others [Ren J, et al. Single-cell sequencing reveals that endothelial cells, EndMT cells and mural cells contribute to the pathogenesis of cavernous malformations. Exp Mol Med. 2023; 55(3):628-42; Zamani M, et al. Single-Cell Transcriptomic Census of Endothelial Changes Induced by Matrix Stiffness and the Association with Atherosclerosis. Adv Funct Mater. 2022;32(47); Depuydt M A C, et al. Microanatomy of the Human Atherosclerotic Plaque by Single-Cell Transcriptomics. Circ Res. 2020; 127(11):1437-55;](FIG. 8A, FIG. 16 and FIG. 17). Among the EC clusters, mRECs showed the majority of DEGs between the two genotypes (58 genes with adjusted P value <0.05), while the numbers of DEGs for cRECs and EndMT-RECs were more limited (2 and 12 genes respectively). GSEA analysis of DEGs in mRECs identified Hypoxia and Glycolysis as the top two significantly enriched Hallmark pathways followed by MTORC1 signaling and Epithelial Mesenchymal Transition pathways in PHD^{TiEC} compared to Cre⁻ mouse (FIG. 8B). Consistent with enhanced glycolytic activity, the glycolysis related genes solute carrier family 2 (facilitated glucose transporter), member 1 (Scl2a1), glucose-6-phosphate isomerase 1 (Gpi1), phosphofructokinase, liver, B-type (Pfkl), aldolase A, fructose-bisphosphate (Aldoa), triosephosphate isomerase 1 (Tpi1), phosphoglycerate kinase 1 (Pgk1), enolase 1, alpha non-neuron (Eno1), pyruvate kinase, muscle (Pkm), lactate dehydrogenase A (Ldha) and solute carrier family 16, member 3 (Slc16a3) were significantly upregulated in mRECs of PHD^{TiEC} IR kidney compared to Cre⁻ control (FIG. 8C).

To examine whether this metabolic profile in ECs was relevant to human AKI, we analysed publicly available single nucleus RNA sequencing (snRNA-seq) data from kidney tissues of patients with severe AKI [Hinze C, et al. Single-cell transcriptomics reveals common epithelial response patterns in human acute kidney injury. Genome Med. 2022;14(1):103]. Hallmark analysis showed Hypoxia and Glycolysis among the top enriched pathways in ECs of human kidneys with AKI (FIG. 8D). Specifically, we found upregulation of glucose-6-phosphate isomerase (GPI), triosephosphate isomerase 1 (TPI1), glyceraldehyde-3-phosphate dehydrogenase (GAPDH), phosphoglycerate kinase 1 (PGK1), phosphoglycerate mutase 1 (PGAM1), enolase 1 (ENO1), pyruvate kinase M1/2 (PKM), lactate dehydrogenase A (LDHA), and solute carrier family 16 member 3 (SLC16A3) in ECs from kidney tissues of patients with AKI compared to the control group (FIG. 8E). Overall, these results demonstrate that post-ischemic endothelial inactivation of PHDs activates the hypoxia response leading to increased endothelial glycolysis, a metabolic response that is also observed in patients with severe AKI.

Post-ischemic endothelial inactivation of PHD1, PHD2 and PHD3 induces EC derived pro-inflammatory responses and exacerbates macrophage accumulation

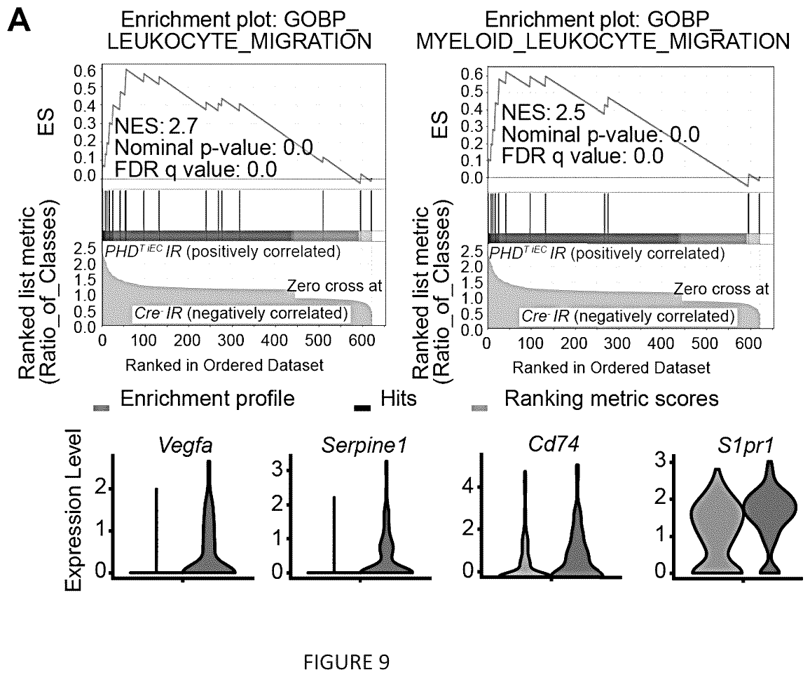

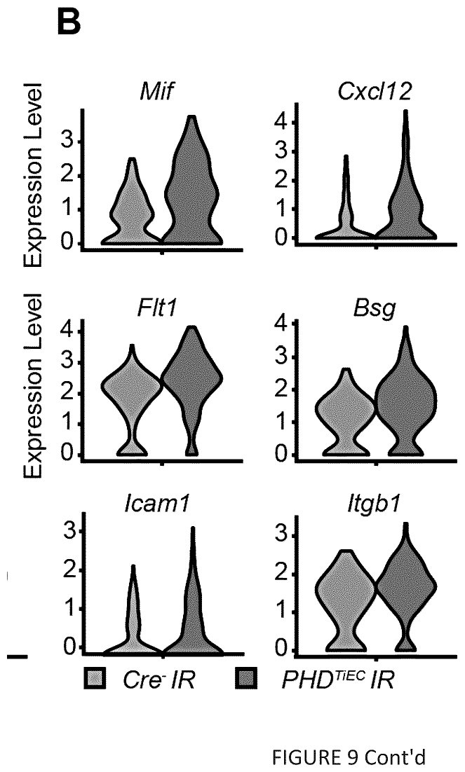

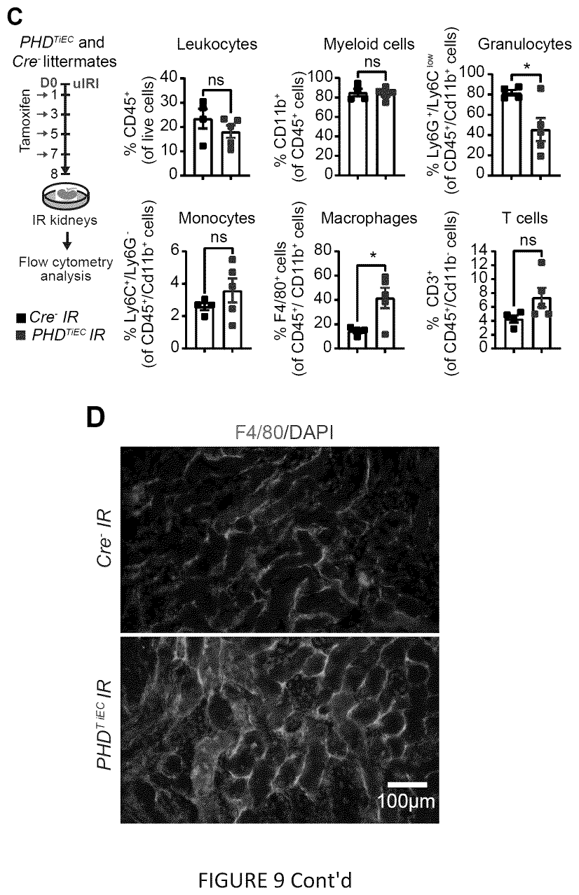

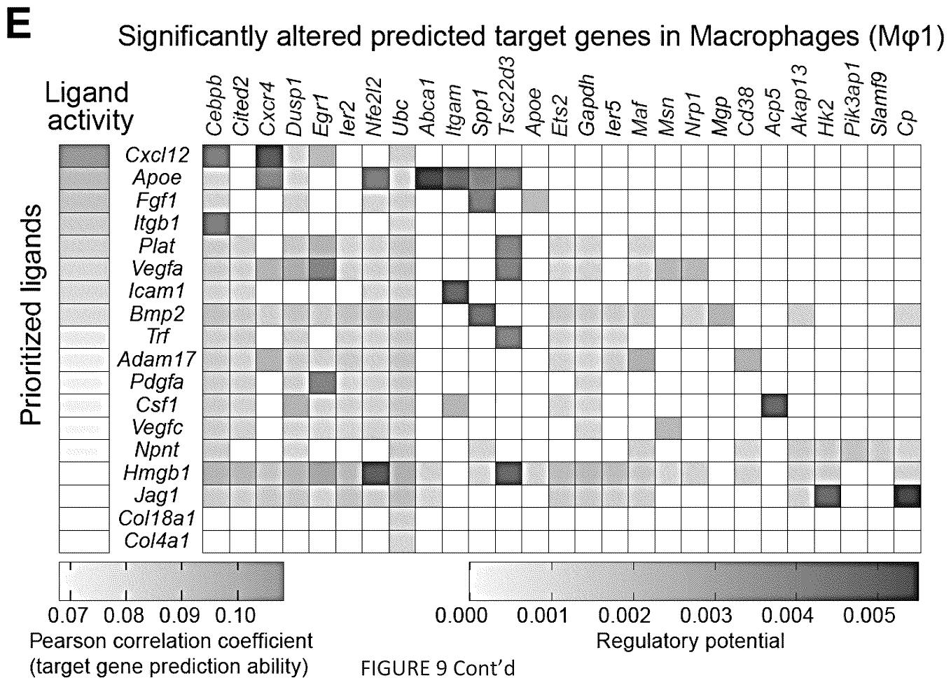

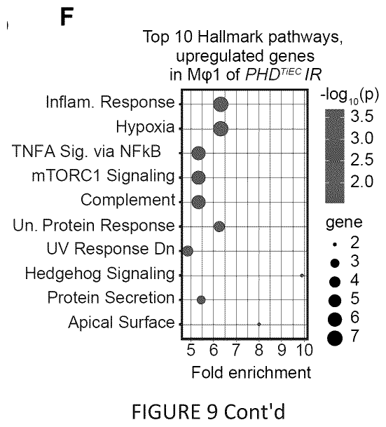

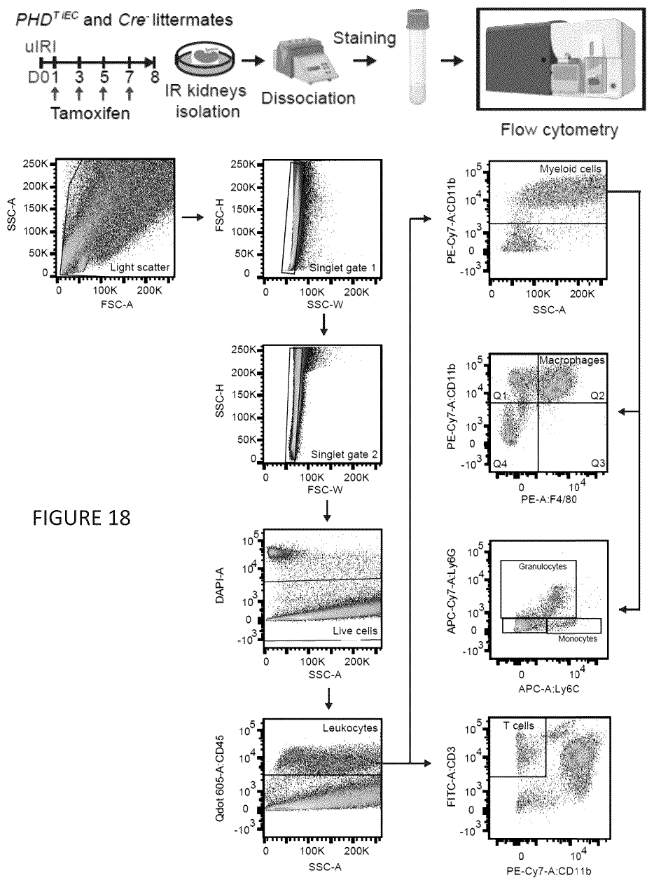

GSEA analysis of DEGs is mRECs showed significant enrichment of Gene Ontology Biological Process (GOBP) gene sets of Leukocyte Migration and Myeloid Leukocyte Migration. These gene signatures were driven by upregulation of macrophage migration inhibitory factor (Mif), C-X-C motif chemokine ligand 12 (Cxcl12), FMS-like tyrosine kinase 1 (Flt1), basigin (Bsg), vascular endothelial growth factor A (Vegfa), serine (or cysteine) peptidase inhibitor, clade E, member 1 (Serpine1), CD74 antigen (Cd74), sphingosine-1-phosphate receptor 1 (S1pr1), intercellular adhesion molecule 1 (Icam1), and integrin beta 1 (Itgb1) in PHD^{TiEC} IR kidney compared to control (FIGS. 9A and B). To characterize immune cell alterations in the post-ischemic kidney microenvironment in the context of endothelial PHD inactivation we performed flow cytometric analysis (FIG. 18). Because at day 14 post uIRI, there was an established maladaptive repair phenotype, we focused our analysis on day 8, an earlier time point that may reveal changes in immune responses contributing to the observed phenotype. Notably, day 8 post-ischemic kidneys of PHD^{TiEC} showed a 2.9-fold increase in macrophages (CD45⁺ CD11b⁺ F4/80⁺) (FIGS. 9C and D) compared to controls. F4/80 immunostaining showed diffuse and variable distribution within the post-ischemic kidneys of PHD^{TiEC} mutants (FIG. 9D). Taken together, these data support that endothelial post-ischemic PHD inactivation promotes maladaptive inflammatory responses and increased macrophage infiltration.

To investigate how mRECs interact with macrophages, we employed NicheNet, a previously published algorithm, which utilizes expression data to infer the potential impact of ligand-receptor interactions on specific targets by integrating existing knowledge of signaling and regulatory networks [Browaeys R, et al. NicheNet: modeling intercellular communication by linking ligands to target genes. Nat Methods. 2020; 17(2):159-62]. We assigned mRECs as “senders” and the different macrophage clusters as “receivers”. NicheNet analysis showed that ligands expressed by mRECs could explain 18 out of the 167 DEGs found in Mφ1 cluster of PHD^{TiEC} mutant compared to control, while the impact was more limited for the other macrophage clusters (2/64 for Mφ2; 5/32 for Mφ3; Mφ4 and pMφ showed no interactions) (FIG. 9E). Several notable ligands and target genes emerged from analysing the interaction of mRECs with Mφ1 cluster. One prominent example is the CXC chemokine CXCL12, which exhibited high expression in mRECs of PHD^{TiEC} mutant. CXCL12 is a known HIF-target gene that can interact with the G protein-coupled chemokine receptor CXCR4, which is abundantly expressed in macrophages. This interaction has the potential to modulate macrophage activation contributing to pro-fibrotic function. Pathway analysis by GSEA identified several Hallmark pathways significantly enriched in Mφ1 cluster of PHD^{TiEC} mutant. These pathways included Hypoxia, Inflammatory response, TNFA signaling via NFkB, mTORC1 signaling, Complement, and Unfolded Protein Response (FIG. 9F). Taken together, these results suggest that post-ischemic endothelial PHD inactivation triggers an endothelial driven pro-inflammatory milieu with macrophages, which may contribute to tissue injury and fibrosis following kidney IRI.

Treatment with the MCT4 Inhibitor Syrosingopine Restores Adaptive Repair in Post-Ischemic Kidney Injury of PHD^{TiEC} Mice

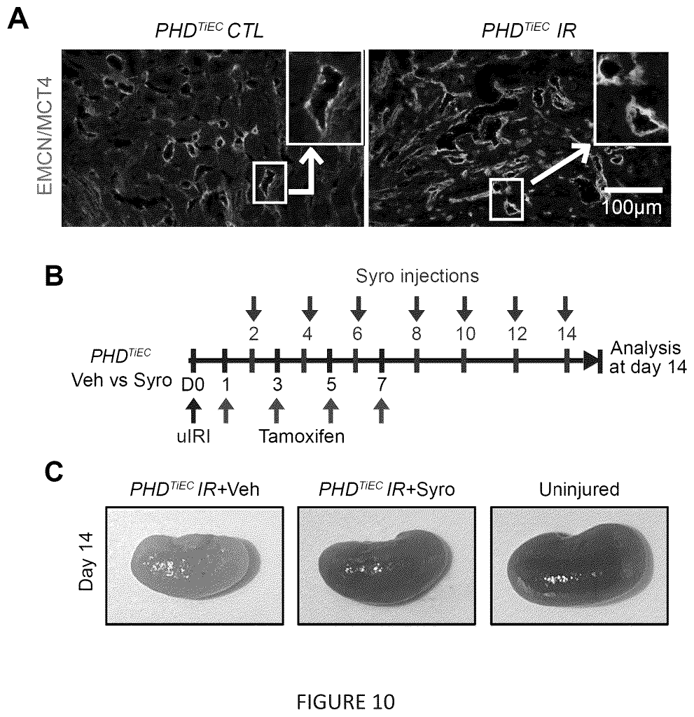

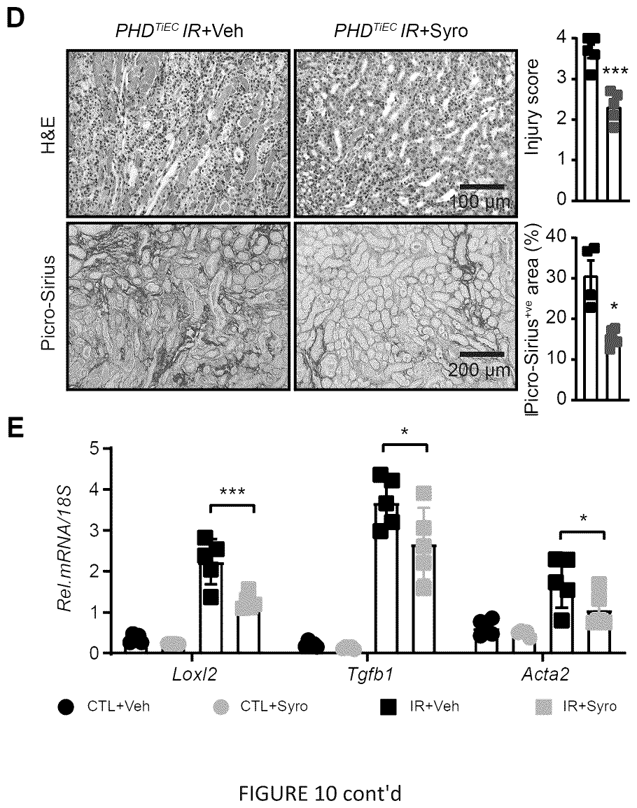

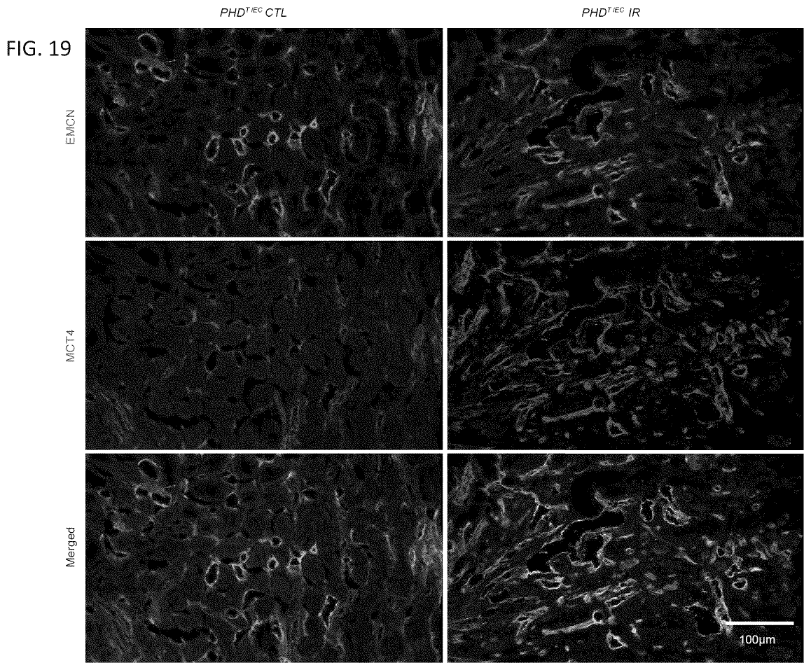

As the primary metabolic response of the post-ischemic endothelium to HIF stabilization in the context of PHD inactivation was increased glycolysis, we next focused on this aspect. While increased lactate generation and efflux via the transporter MCT4 represent the final steps of hypoxia-induced glycolysis, lactate export was recently shown to be among the four key steps controlling glycolytic flux. Slc16a3, the gene encoding MCT4 was significantly increased in mRECs of post-ischemic PHD^{TiEC} kidney as well as in ECs of human kidneys with severe AKI. Indeed, immunofluorescence staining confirmed high expression of MCT4 protein in the post-ischemic kidney endothelium of PHD^{TiEC} (FIG. 10A and FIG. 19). We next sought to investigate whether increased endothelial MCT4 causally contributes to the maladaptive post-ischemic kidney repair observed in the PHD^{TiEC} mice. To this end, we used syrosingopine, a dual inhibitor of monocarboxylate transporters MCT1 and MCT4 (60-fold higher potency on MCT4), which leads to end-product inhibition of glycolysis. As depicted in FIG. 10B, PHD^{TiEC} mice were treated with syrosingopine or vehicle starting on day 2 after uIRI and then every other day until day 14 post uIRI. Strikingly, we found that treatment with syrosingopine significantly suppressed tubular injury and fibrosis as indicated by ˜37% and ˜51% reduction in injury scoring and collagen accumulation, respectively compared to the vehicle-treated mice (n=4-6, P<0.05) (FIGS. 10C and D). Furthermore, the mRNA levels of profibrotic genes Loxl2, Tgfb1 and Acta2 were significantly downregulated in syrosingopine treated group compared to vehicle treated group (n=5, P<0.05) (FIG. 10E). Taken together, our studies demonstrate that inhibition of endothelial MCT4 by syrosingopine restores adaptive kidney repair following IRI in PHD^{TiEC} mutants. MCT4 inhibition diminishes endothelial derived pro-inflammatory responses induced by hypoxia-reoxygenation and IL-1

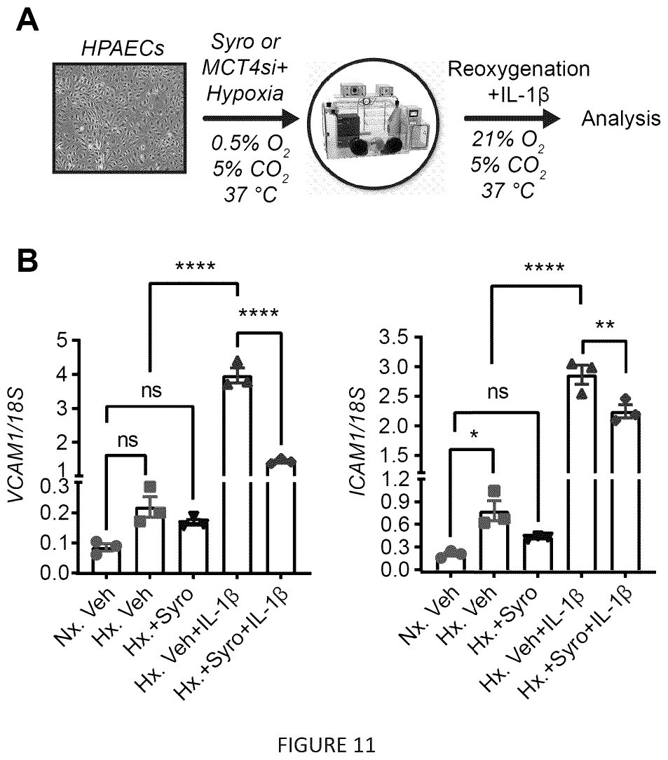

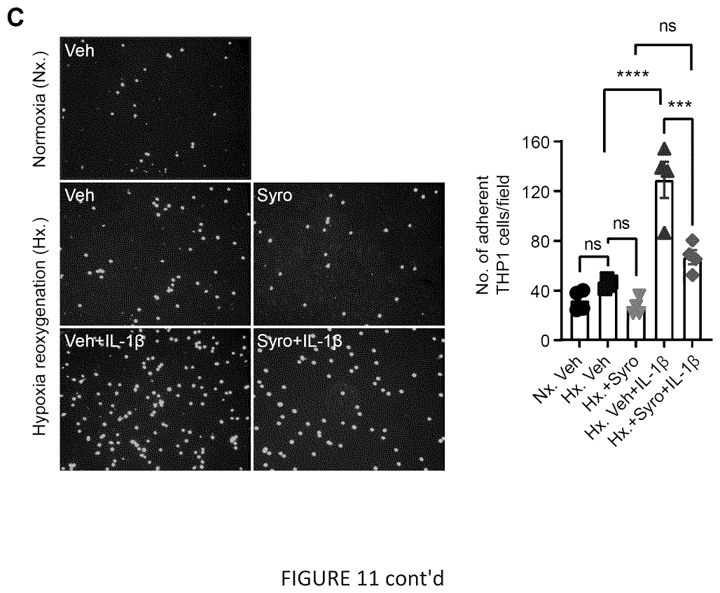

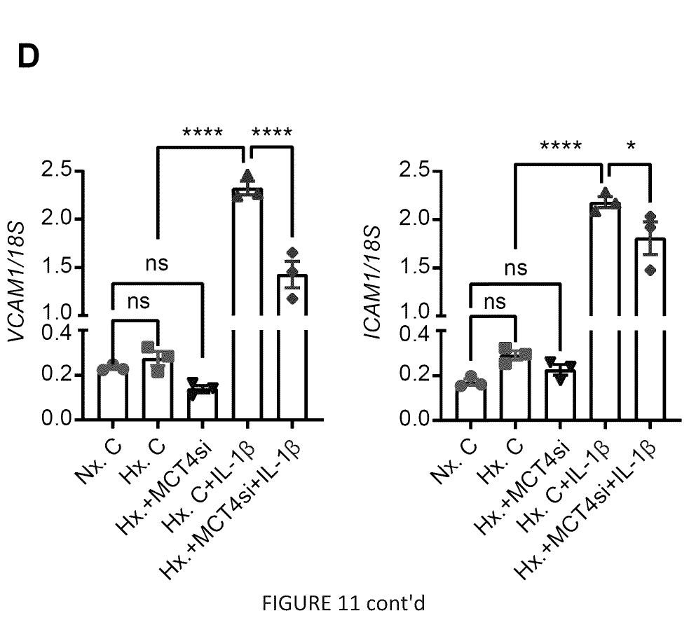

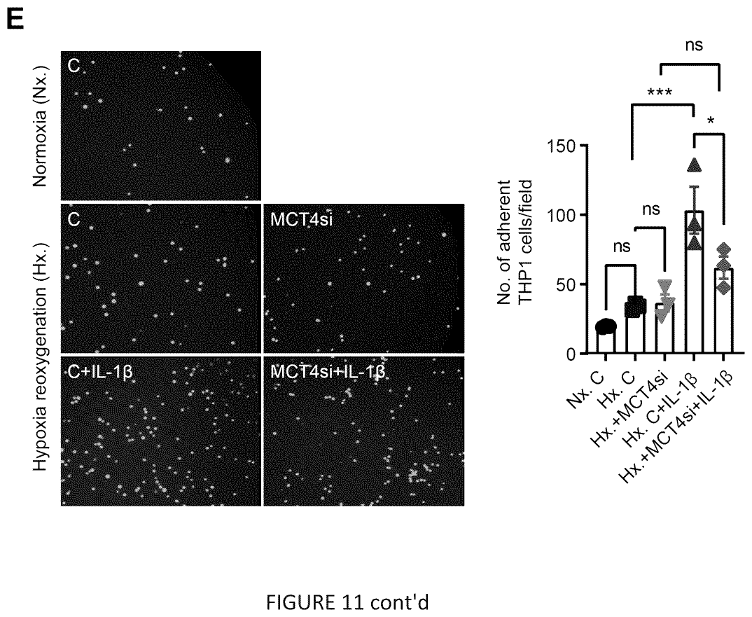

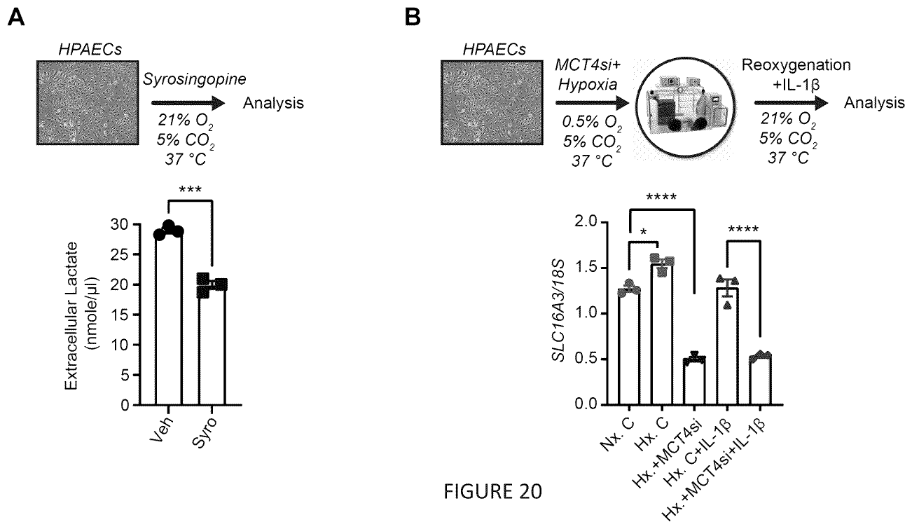

To assess whether MCT4 inhibition acts cell-autonomously in ECs to suppress pro-inflammatory responses, we performed in vitro experiments in which human primary pulmonary artery endothelial cells (HPAECs) were activated by 18 hours exposure to hypoxia (0.5% O₂), followed by reoxygenation for 8 hours in the presence of IL-1β treatment (1 ng/mL) (FIG. 11A). To inhibit MCT4, we first used syrosingopine at concentration of 5 μM, which diminished lactate secretion, as indicated by reduction in extracellular lactate levels (FIG. 20A). We found that stimulation of HPAECs by hypoxia-reoxygenation and IL-1β led to ˜23- and ˜30-fold increase of vascular cell adhesion molecule 1 (VCAM1) and intercellular adhesion molecule 1 (ICAM1), respectively, while treatment with syrosingopine significantly suppressed the effects of hypoxia-reoxygenation and IL-1β on the expression of EC adhesion molecules (FIG. 11B). Furthermore, under the same experimental conditions, treatment with syrosingopine significantly reduced adhesion of monocytes (THP-1 cells) to HPAECs by ˜48% (FIG. 11C). Consistent responses were observed when we used siRNA against MCT4. With ˜67% knock down efficiency (FIG. 20B), we observed that MCT4siRNA significantly suppressed VCAM1 and ICAM1 transcripts in ECs stimulated by hypoxia-reoxygenation and IL-10 compared to negative control siRNA, reducing adhesion of monocytes to HPAECs by ˜40%. (FIGS. 11D and E). Taken together, these results demonstrate that MCT4 inhibition suppresses EC interaction with monocytes in a cell autonomous manner, an effect that contributes to favorable effects exerted by syrosingopine, when given post kidney IRI.

### Discussion

The evidence shows that the regulation of endothelial oxygen sensing signaling has critical implications in post-ischemic kidney repair. Within the kidney endothelium, we demonstrate the expression of PHD1 and PHD3 isoforms along with PHD2 and show their critical contribution in regulating HIF signaling in the renal vascular bed. By investigating the outcomes of kidney repair in different conditional knock out strains, we provide evidence that concurrent loss of the three PHD isoforms and not PHD2 alone dictates post-ischemic kidney repair. We furthermore identify the endothelial glycolysis/MCT4 axis as a promising therapeutic target. Pharmacological inhibition of MCT4 holds the potential to interrupt the transition from AKI to CKD by mitigating inflammation.

PHD enzymes are non-heme iron-dependent dioxygenases that catalyze the four-electron reduction of O₂. Specifically, they incorporate each O₂ atom, respectively, into the tricarboxylic acid (TCA) cycle metabolite 2-oxoglutarate (2OG) and the substrate polypeptide, forming succinate, carbon dioxide, and trans-4-hydroxylated prolyl products. With Km values falling within the 230-250 μM range, PHDs are sensitive to fluctuations in local O₂ in tissues, thus functioning as oxygen sensors. While PHD2 is considered the main oxygen sensor, the other isoforms have been implicated in regulating hypoxia signaling. Consistently, we have demonstrated an overlapping pattern of expression for PHDs in kidney endothelium, which probably allows significant compensatory responses. This may explain the lack of HIF stabilization when endothelial PHD2 was individually deleted in contrast to the robust HIF activation we observed when the three PHD isoforms were concurrently inactivated. While the regulation of HIF by PHDs in different vascular beds requires further investigation, our findings are particularly relevant for IRI, where significant hypoxia could limit the activity of all PHD isoforms. Furthermore, because PHDs are regulated not only by oxygen but also by factors such as 20G, oxidative stress and abnormal concentrations of endogenous metabolites, PHD mediated hydroxylation can be altered even under normoxia, creating a “pseudo-hypoxic state”. For example, succinate, a TCA cycle metabolite known to accumulate with IRI can inhibit all three PHDs. Therefore, various pathophysiological signals within the post-ischemic kidney microenvironment can inhibit endothelial PHDs dictating tissue remodeling. However, it remains unclear how PHDs in different cell types respond to hypoxic and pseudo-hypoxic signals and how the integrated responses alter kidney repair. Although it may be challenging, additional genetic studies involving inducible deletion of PHDs in different cell types are required to define the role of PHD/HIF pathway in the context of kidney repair.

Capillary rarefaction was exacerbated after ischemic kidney injury in the setting of post-ischemic endothelial PHD inactivation and was associated with the presence of partially “dedifferentiated” ECs as reflected by the EC-EndMT cluster revealed by ScRNA-seq. EndMT could be induced directly by HIF activation in the context of endothelial PHD inactivation as indicated by previous studies linking HIFs to EndMT in pulmonary and cardiac fibrosis by upregulating the transcription factor SNAI1. While the contribution of EndMT in our model is unclear, impaired angiogenic capacity could represent another EC autonomous mechanism by which PHD inactivation leads to capillary drop-out, as we and others recently showed in cultured ECs. Regardless of the underlying mechanism, by inducing kidney tissue hypoxia, capillary rarefaction can function as an upstream regulator of various defective responses. For example, Lovisa et al. showed an intriguing link between endothelial dysfunction and impaired tubular epithelial metabolism shifting from fatty acid oxidation to glycolysis [Lovisa S, et al. Endothelial-to-mesenchymal transition compromises vascular integrity to induce Myc-mediated metabolic reprogramming in kidney fibrosis. Sci Signal. 2020;13(635)].

Post-ischemic endothelial PHD inactivation augmented interstitial fibrosis and inflammation following kidney IRI, and these responses were associated with robust HIF activation. Based on previous studies in which constitutive deletion of endothelial HIF-2 enhanced IRI-induced kidney fibrosis and inflammation, and pre-ischemic inactivation of endothelial PHD2 was protective, our findings were unanticipated. These possibilities are of course not mutually exclusive. Pharmacological approaches of PHD inhibition applied prior to ischemic kidney injury have provided renoprotective effects, which though were not observed when HIF signaling was activated during the post-ischemic phase. Our study extends these findings on the timing of PHD inactivation being a critical factor in determining kidney injury responses by employing a genetic approach of inducible EC specific targeting. Furthermore, our system is not confounded by the potential off-target effects of PHD inhibitors on other 2-oxoglutarate-dependent dioxygenases. The observed suppression of fibrosis by inhibiting the HIF-1 regulated lactate transporter MCT4 supports the notion that glycolysis driven by persistent endothelial HIF-1 activation contributes to post-ischemic maladaptive kidney repair. Interestingly, Lim et al. reported that MCT4 downregulation alone significantly inhibits HIF transcriptional activity through a feed-back loop signaling [Lim K S, et al. Inhibition of monocarboxylate transporter-4 depletes stem-like glioblastoma cells and inhibits HIF transcriptional response in a lactate-independent manner. Oncogene. 2014; 33(35):4433-41], which could represent an additional mechanism underlying the renoprotective effects of syrosingopine. Nevertheless, it is plausible that non-HIF hydroxylation substrates of PHD could contribute to the observed outcomes by PHD inhibition. For example, PHD inhibition could reduce the hydroxylation of IKKβ derepressing NFkB signaling, a response known to induce endothelial adhesion molecules binding and transmigration of leukocytes.

ECs are known to use glycolysis to meet >80% of their ATP demands, preserving oxygen for perivascular cells. Through scRNA-seq analysis, our study now unveiled that glycolysis can be further enhanced in post-ischemic renal ECs of PHD^{TiEC} mice, which is in agreement with the metabolomic changes we recently reported in ECs treated with the pan-PHD inhibitor, dimethyloxalylglycine (DMOG). Importantly, increased endothelial glycolysis was associated with pro-inflammatory changes that lead to enhanced macrophage infiltration. In accordance with our findings, recent studies reported induction of glycolysis in ECs subjected to the inflammatory mediators TNFA and lipopolysaccharide, both known to induce HIF activation. In this setting, among the significantly upregulated glycolytic genes, PFKFB3 was as identified a key regulator of this metabolic reprogramming. Notably, genetic or pharmacologic inactivation of PFKFB3 abrogated inflammation in various disease models such as LPS-induced inflammation and pulmonary hypertension demonstrating that PFKFB3-driven glycolysis drives endothelial pro-inflammatory pathways. Consistent with the endothelial glycolytic signature observed in ECs in our scRNA-seq data, Pfkfb3 showed a trend for upregulation, which though did not reach statistical significance probably due to limited power of our analysis. Importantly, by analyzing publicly available scRNA-seq data from kidneys of patients with severe AKI, we demonstrate that augmented endothelial glycolysis is observed in the human AKI endothelium along with hypoxia and inflammatory pathways such as IFN-γ, IFN-α and TNFA. Thus, our findings suggest a novel role for PHD/HIF in regulating immunometabolic phenotypes of kidney endothelium.

The HIF-1 induction of MCT4 allows the efficient export of lactic acid and enables glycolytic tumors to grow within an acidic microenvironment. Furthermore, because MCT4-mediated lactic acid transport can regulate glycolytic flux, blocking MCT4 has been proposed as a strategy to suppress tumor growth in different cancers by disrupting the Warburg effect. Given that a) ECs resemble cancer cells in their preferential use of glycolysis b) MCT4 was significantly upregulated by endothelial PHD inactivation in the post-ischemic kidney, we postulated that MCT4 inhibition might promote adaptive kidney repair by suppressing endothelial glycolysis. To test this hypothesis, we treated PHD^{TiEC} mice with syrosingopine starting on day 2 after uIRI and found that MCT4 inhibition remarkably reduced the tubular injury and development of fibrosis at day 14 post uIRI. To assess whether MCT4 inhibition had EC autonomous effects, HPAECs activated to a pro-inflammatory phenotype by hypoxia-reoxygenation and IL-10, were treated with syrosyngopine or siMCT4. Both genetic silencing and pharmacologic MCT4 inhibition significantly suppressed the expression of EC-adhesion molecules reducing the adhesion of THP1 cells. Interestingly, inhibition of CD147, a broadly expressed transmembrane glycoporotein that is essential for MCT4 function, has been reported to reduce post-ischemic brain inflammatory cell infiltration by reduced NFkB activation in brain microvascular ECs. While this mechanism could explain our findings, a broader reprogramming of EC metabolism by MCT4 inhibition may be a key driver. For instance, Cluntun et al showed that MCT4 inhibition shifted glucose flux from lactate to citrate and TCA cycle in cardiomyocytes [Cluntun A A, et al. The pyruvate-lactate axis modulates cardiac hypertrophy and heart failure. Cell Metab. 2021; 33(3):629-48.e10]. Further studies are needed to investigate how metabolic reprogramming by MCT4 inhibition is linked to endothelial function.

In summary, our genetic studies demonstrate that post-ischemic endothelial PHD inactivation leads to HIF activation and maladaptive kidney repair, which can be reversed by MCT4 inhibition.

### Methods

### Generation of Mice, Genotyping, and Animal Procedures

The generation and genotyping of Phd1 (Egln2), Phd2 (Egln1), and Phd3 (Egln3) floxed mice have been described previously (27, 28, 76)[Minamishima Y A, et al. Somatic inactivation of the PHD2 prolyl hydroxylase causes polycythemia and congestive heart failure. Blood. 2008; 111(6):3236-44; Takeda K, et al. Placental but not heart defects are associated with elevated hypoxia-inducible factor alpha levels in mice lacking prolyl hydroxylase domain protein 2. Mol Cell Biol. 2006; 26(22):8336-46; Tomita S, et al. Conditional disruption of the aryl hydrocarbon receptor nuclear translocator (Arnt) gene leads to loss of target gene induction by the aryl hydrocarbon receptor and hypoxia-inducible factor lalpha. Mol Endocrinol. 2000; 14(10):1674-81]. For inducible endothelial-specific inactivation of the floxed allele, we used the Cdh5(PAC)-CreER^{T2} transgenic mouse line, provided by Dr. Ralph Adams [Sorenson, I., (supra)]. To examine recombination efficiency in the post-ischemic kidney, we generated Cdh5(PAC)CreER^{T2}; mT/mG mice by crossing the ROSA26-ACTB-tdTomato,-EGFP reporter mice (JAX stock number 007576) to the Cdh5(PAC)CreER^{T2} transgenic mouse line. Cre-mediated inactivation was induced by four intraperitoneal (IP) injections of tamoxifen dissolved in 10% ethanol in corn oil at 20 mg/mL (3 mg/mouse) given every other day. Mice were maintained in a specific pathogen-free facility on a 12-hour light/12-hour dark cycle. Littermates were randomly assigned to experimental groups.

Male mice 8-10 weeks of age underwent renal IRI surgery as previously described [Torosyan R, et al. Hypoxic preconditioning protects against ischemic kidney injury through the IDO1/kynurenine pathway. Cell Rep. 2021;36(7):109547]. Anesthesia was induced by IP injection of xylazine (10 mg/kg IP) and ketamine (90-120 mg/kg IP). After making a small midline abdominal incision, the left renal pedicle was occluded by applying a microaneurysm clamp for 25 minutes, while the right kidney was kept intact as an internal control in the unilateral IRI. For bilateral renal IRI, clamps were applied in both renal pedicles for 23 minutes. After the specified time, the clamps were removed, and reperfusion was visually confirmed. The incision site was closed with a 6-0 suture followed by the closure of the skin wound with Michel miniature clips. Throughout the surgical procedure, body temperature was monitored by a rectal probe and controlled with a heating pad at 37° C.

For in vivo MCT4 inhibition studies, mice were treated with syrosingopine (7.5 mg/kg) or vehicle starting on day 2 post IRI every other day until dissection on day 14 post IRI.

### DNA, RNA, and Protein Analyses

DNA and RNA were extracted and used for genomic or real-time PCR analysis, as previously described [Majmundar A J, et al. Hypoxia-Inducible Factors and the Response to Hypoxic Stress. Mol Cell. 2010; 40(2):294-309]. Mouse and human primer sequences are listed in Tables 1 and 4. Real-time PCR was run in the QuantStudio 3 Real-Time PCR system (Applied Biosystems) using SYBR green or TaqMan PCR Master Mix. 18S rRNA was used for normalization.

TABLE 4
Primer sequences. Shown are sequences
of primer sets used for the expression
analysis of the indicated mouse and
human genes by RT-PCR.

| | | |
|---|---|---|
| Gene | Forward Primer | Reverse Primer |

Mouse

| | | |
|---|---|---|
| Loxl2 | SEQ ID NO: 1 | SEQ ID NO: 2 |
| | 5′-GATCTTCAGCCC | 5′-CAAGGGTTGCTC |
| | CGATGGA-3′ | TGGCTTGT-3′ |
| Tgfb1 | SEQ ID NO: 3 | SEQ ID NO: 3 |
| | 5′-TGGCGAGCCTTA | 5′-TCGACATGGAGC |
| | GTTTGGA-3′ | TGGTGAAA-3′ |
| Acta2 | SEQ ID NO: 5 | SEQ ID NO: 6 |
| | 5′-CCTGACGCTGAA | 5′-TTTTCCATGTCG |
| | GTATCCGATAG-3′ | TCCCAGTTG-3′ |
| Kim1 | SEQ ID NO: 11 | SEQ ID NO: 12 |
| | 5′-AAACCAGAGATT | 5′-GTCGTGGGTCTT |
| | CCCACACG-3′ | CCTGTAGC-3′ |

Human

| | | |
|---|---|---|
| SLC16A3 | SEQ ID NO: 13 | SEQ ID NO: 14 |
| | 5′-GGGTGGGAACCG | 5′-CTTGCGGCTTGG |
| | TGTCATT-3′ | CTTCA-3′ |
| VCAM1 | SEQ ID NO: 15 | SEQ ID NO: 16 |
| | 5′-GCTTCAGGAGC | 5′-AAGGATCACGAC |
| | TGAATACCC-3′ | CATCTTCC-3′ |
| ICAM1 | SEQ ID NO: 17 | SEQ ID NO: 18 |
| | 5′-CCACAGTCACCT | 5′-AGTGTCTCCTG |
| | ATGGCAAC-3′ | GCTCTGGTT-3′ |

Nuclear protein was extracted using NE-PER Nuclear and Cytoplasmic Extraction Reagents (Thermo Fisher Scientific), following the provided instructions. The extracted nuclear protein samples were then subjected to separation on an SDS-PAGE gel, transferred on a nitrocellulose membrane, and subsequently incubated with HIF-1α antibodies (Cayman) or HIF-2a antibodies (Novus Biologicals) at 4° C. Following an overnight incubation, the nitrocellulose membrane was washed and incubated with a secondary antibody (Novus Biologicals) for 90 minutes at 4° C. Chemiluminescent signal detection was performed using SuperSignal™ West Femto Chemiluminescent Substrate (Thermo Fisher Scientific), and membranes were imaged on the iBright Imaging System (Thermo Fisher Scientific).

### Histopathological and Immunofluorescence Analysis

For the histological examination, kidneys were fixed in 10% formalin buffer and paraffin blocks were prepared. To assess tubular damage and interstitial fibrosis, 5-μm transverse plane kidney sections were separately stained with hematoxylin and eosin (H&E) and Picro-Sirius red. Tubular injury was semi-quantitatively scored by determining the percentage of tubules in the corticomedullary junction that displayed necrosis, loss of brush border, cast formation, and tubular dilatation (0, unaffected; 1, 1%-25%; 2, 26%-50%; 3, 51%-75%; 4, 76%-100%) (78). Fibrosis was assessed by the percentage of Picro-Sirius red^{+ve} area determined with ImageJ software (http://rsbweb.nih.gov/ij). For tubular injury scoring and quantification of Picro-Sirius red^{+ve} area ³10 and ³5 random visual fields of corticomedullary region, respectively were analyzed per kidney section for each sample at a magnification of ×200.

Immunofluorescence staining was performed using primary antibodies against PHD1 (Abcam; Catalog #ab113077), PHD2 (Novus Biologicals; Catalog #NB100-137), PHD3 (Novus Biologicals; Catalog #NB100-139), Endomucin (Abcam; Catalog #ab106100), F4/80 (Abcam; Catalog #ab6640), and MCT4 (Proteintech; Catalog #22787-1-AP). Goat anti-rat Alexa Fluor® 488 (Invitrogen; Catalog #A11006), Goat anti-rabbit Alexa Fluor® 594 (Invitrogen; Catalog #A32740), and Goat anti-rabbit Alexa Fluor® 647 (Invitrogen, Catalog #A32733) were used as secondary antibodies. VECTASHIELD Vibrance Antifade Mounting Medium with DAPI (Vector Laboratories, Inc. Catalog #NC1601055) was used as mounting media. Images were captured by a fluorescence microscope (Nikon Ti2 widefield microscope) and analyzed using Fiji software (Image J). Percentage of Endomucin^{+ve}area was determined with ImageJ software.

### BUN and GFR Measurements

Serum BUN levels were measured using the QuantiChrom Urea Assay Kit (BioAssay Systems) following the manufacturer's instructions. Transcutaneous GFR was measured via the transcutaneous clearance of FITC-Sinistrin using a miniaturized fluorescence detector (NIC-Kidney device) on day 14 post bIRI[Schock-Kusch D, et al. Reliability of transcutaneous measurement of renal function in various strains of conscious mice. PLoS One. 2013;8(8):e71519; Friedemann J, et al. Improved kinetic model for the transcutaneous measurement of glomerular filtration rate in experimental animals. Kidney Int. 2016; 90(6):1377-85].

### Preparation of Single-Cell Suspension

Injured kidneys from PHD^{TiEC} and Cre⁻ mice were collected on day 14 post uIRI. Single-cell suspensions were prepared using Multi Tissue Dissociation Kit 2 (Miltenyi Biotec; Catalog #130-110-203) following the instructions provided with the kit. Briefly, IR kidneys were cut into 6-8 pieces using single edge blades and placed in gentleMACS C tube with 5 ml dissociation buffer (4.8 mL of Buffer X, 50 μL of Enzyme P, 50 μL of Buffer Y, 100 μL of Enzyme D, and 20 μL of Enzyme A provided in Multi Tissue Dissociation Kit 2). GentleMACS C tubes were placed in a gentleMACS Dissociator (Miltenyi Biotec) and kidneys were dissociated using program Multi_E_01. Dissociated kidneys were incubated for 30 minutes at 37° C. with continuous rotation using the MACSmix Tube Rotator (Miltenyi Biotec). GentleMACS C tubes were then placed in the gentleMACS Dissociator and Multi_E_02 program was run. GentleMACS C tubes were detached and 10 mL neutralizing buffer (1×PBS with 2% fetal bovine serum) was added to stop dissociation reaction. Cell suspension was filtered through 100 μm and then 30 μm cell strainer. Cell suspension was centrifuged at 400 g for 10 minutes. Cell pellet was incubated with 1 mL of RBC lysis buffer on ice for 1 minute and after the addition of 10 mL neutralizing buffer, cells were recentrifuged at 400 g for 5 minutes. Cells were suspended in 10 mL neutralizing buffer and centrifuged again at 100 g for 5 minutes. Finally, cell pellet was suspended in 1×PBS with 0.04% BSA and filtered through 30 μm. Cell number and viability were analyzed using the Nexcelom Cellometer Auto 2000 with the AOPI fluorescent staining method. This method resulted in single-cell suspensions with >80% viability.

scRNA-Seq Library Generation and Sequencing

Sixteen thousand cells were loaded into the Chromium Controller (10× Genomics; Catalog #PN-120223) on a Chromium Next GEM Chip G (10× Genomics, Catalog #PN-1000120) and processed to generate single-cell gel beads in the emulsion (GEM) according to the manufacturer's protocol. The cDNA and library were generated using the Chromium Next GEM Single Cell 3′ Reagent Kits v3.1 (10× Genomics; Catalog #PN-1000286) and single Index Kit T Set A (10× Genomics; Catalog #PN-1000213) according to the manufacturer's manual. For the cell hashing library, it was constructed according to Biolegend's published protocol on HTO library preparation guidelines with single index adapters. Quality control for the constructed library was performed by the Agilent Bioanalyzer High Sensitivity DNA Kit (Agilent Technologies; Catalog #5067-4626), while Qubit DNA HS Assay Kit was used for qualitative and quantitative analysis. The multiplexed libraries were pooled and sequenced on an Illumina HiSeq 4000 sequencer with 2×50 paired-end kits using the following read lengths: 28 bp for Read1 for cell barcode and UMI, and 90 bp for Read2 for transcript. Raw sequencing data in base call format (.bcl) was demultiplexed using Cell Ranger from 10× Genomics, converting the raw data into FASTQ format. Cell Ranger was also used for alignment of the FASTQ files to the mouse reference genome (mm10) and to count the number of reads from each cell that align to each gene.

scRNA-Seq Data Analysis

The resulting matrix files, which summarize the alignment results, were imported into Seurat (Satija Lab, NYGC) for further analysis. In Seurat (v4.3.0), each individual sample was transformed to Seurat object. Only genes expressed in 3 or more cells, and cells expressing at least 200 genes, or more were used for further analysis. Further, cells with <500 unique molecular identifier (UMI) counts (cell fragments) and >100,000 UMI counts (potentially cell duplets) were excluded [Xu L, et al. Immune-mediated tubule atrophy promotes acute kidney injury to chronic kidney disease transition. Nat Commun. 2022;13(1):4892.]. Cells with mitochondrial gene percentage over 50% and low complexity cells such as red blood cells with <0.8 log 10 genes per UMI counts were also excluded from the analysis. Merged data were normalized, scaled, and principal component analysis (PCA) was done. The top 20 principal components were chosen for neighbors and cell clustering performed with resolution of 0.8. The cell clusters were visualized in two-dimensional space by the Uniform Manifold Approximation and Projection (UMAP). Both genotypes showed similar clustering and cell populations in separate and overlapping DimPlot [Thomson B R, et al. Cellular crosstalk regulates the aqueous humor outflow pathway and provides new targets for glaucoma therapies. Nat Commun. 2021;12(1):6072]. To identify cell clusters, marker genes were assessed using “FindAllmarkers” function of Seurat with the setting of min.pet of 0.25 and logfc.threshold of 0.25. Based on marker analysis, one cluster predominately enriched with lncRNAs Gm26917 and Gm42418 associated with ribosomal RNA contamination was excluded [Liu Y, et al. Single-Cell Profiling Reveals Divergent, Globally Patterned Immune Responses in Murine Skin Inflammation. iScience. 2020;23(10):101582; Akama-Garren E H, et al. Follicular T cells are clonally and transcriptionally distinct in B cell-driven mouse autoimmune disease. Nat Commun. 2021;12(1):6687]. The remaining 28 clusters were annotated based on the expression of top marker genes supported by published studies (Table 2). Differential gene expression analysis was performed, and genes with adjusted P<0.05, log₂FC >0.2 were considered significantly regulated. Gene set enrichment analysis was performed using GSEA software (v4.2.3) and by Enrich® (https://maayanlab.cloud/Enrichr/). Data were deposited in the NCBI's Gene Expression Omnibus database (GEO GSE244064).

Public scRNA-Seq Data Analysis

Murine scRNA-seq data. To examine the expression of genes encoding the three HIF prolyl hydroxylase isoforms (Egln1, Egln2 and Egln3) in mouse kidney ECs, we extracted adult mouse kidney endothelial scRNA-seq data from the EC Atlas database created by the Carmeliet group [Kalucka J. et al, supra]. Data was processed and analyzed using standard Seurat's workflow for data processing and normalization (https://satijalab.org/seurat/articles/pbmc3k_tutorial.html). Cell clusters were generated with 0.7 resolution and identified by analyzing the expression of EC marker genes (Plvap, Igfbp3, S100a6, Sox17, Igfbp7, Aqp1, Pi16, Plat, Ehd3, Cyp4b, and Lpl) [Dumas S J, Meta E, Borri M, Luo Y, Li X, Rabelink T J, et al. Phenotypic diversity and metabolic specialization of renal endothelial cells. Nat Rev Nephrol. 2021; 17(7):441-64].

Human scRNA-seq data. The expression of EGLNs genes in human kidneys ECs was assessed by extracting human kidney biopsy scRNA-seq datasets generated by Kidney Precision Medicine Project (KPMP) and available via Gene Expression Omnibus (GSE140989; Table 2) [Menon, R., et al., supra]. Seurat files were created and combined following Seurat's standard workflow. EC cell cluster was identified based on the expression of endothelial markers EMCN, CD34, PLVAP and EHD3. After extraction, EC were further clustered into 3 EC subclusters, which were identified based on the expression of different EC markers (PLVAP, RGCC, MARCKS, SPARCL1, IGFBP7, TGFBR2, SOST, PLAT, EHD3, MEG3, SERPINE2, KCTD12, LTBP4, CLDN5, and IGF2) [Menon, R. et al., supra].

Human snRNA-seq data from AKI and control kidney tissues. Human snRNA-seq datasets from AKI and control kidney tissues (8 AKI; 6 controls) were extracted from Gene Expression Omnibus (GSE210622) [Hinze C, et al., supra]. Raw data was analyzed using Seurat's best practice workflow for data integration using the reciprocal PCA approach (https://satijalab.org/seurat/articles/integration_rpca.html) with default parameters. Clusters were then analyzed for marker genes and EC cluster (CD34⁺ and EMCN⁺) was extracted, combined, and analyzed following Seurat's best practice standard workflow for data integration and analysis (https://satijalab.org/seurat/articles/integration_introduction.html). Differential gene expression analysis was performed, and significantly upregulated genes (adjusted P<0.01, Log₂FC>0.25) were subjected to hallmark analysis by Enrich® (https://maayanlab.cloud/Enrichr/). The expression levels of glycolytic genes in AKI and control kidney ECs were directly derived from the publicly accessible online interface (https://shiny.mdc-berlin.de/humAKI/) (39).

### Flow Cytometry

Following perfusion with PBS, kidneys were harvested, minced, and incubated in dissociation solution (Multi Tissue Dissociation Kit 2; Miltenyi Biotec; Catalog #130-110-203) for 30 minutes at 37° C. After dissociation, cells were passed through a 70- and 40-μm filter, and 10 mL of cold staining buffer (PBS+2% fetal bovine serum) was added. Cells were centrifuged (400 g for 10 minutes), and cell pellets were resuspended in cold RBC lysis buffer, centrifuged, and resuspended in staining buffer. Following incubation with CD16/32 (Fc block), single-cell suspensions were labeled with different fluorophore-tagged antibodies. The following antibodies were used: CD16/32 (eBioscience™, Catalog #14-0161-85), CD45 (clone 30-F1, Biolegend; Catalog #103140), F4/80 (clone BM8, Biolegend; Catalog #123110), CD11b (clone M1/70, Biolegend; Catalog #101216), CD3 (clone 17A2, Biolegend; Catalog #100210), Ly6C (clone HK1.4; Biolegend; Catalog #128010), and Ly6G (BD biosciences; Catalog #560600), and CD31 (clone 390, Invitrogen; Catalog #17-0311-82).

### Cell Culture

Human primary pulmonary artery ECs (HPAECs) were obtained from ATCC and grown on gelatin-coated dishes in Endothelial Cell Basal Medium-2 (Lonza, Catalog #CC-3156) supplemented with EGM-2 SingleQuots Supplements (Lonza, Catalog #CC-4176). MCT4 siRNA was purchased from Qiagen (FlexiTube GeneSolution GS9123 for SLC16A3; Catalog #1027416). AllStars negative control siRNA (Qiagen) was used as control siRNA. HiPerFect transfection reagent (Qiagen, Catalog #301707) was used for siRNA transfection experiments. For hypoxia experiments, MCT4siRNA transfected- or syrosingopine-treated HPAECs and their corresponding controls (AllStars negative control transfected and vehicle-treated, respectively) were incubated for 18 hours in a hypoxia chamber (Coy Laboratory Products) at 0.5% O₂, 37° C. in a 5% CO₂ humidified environment. During reoxygenation, HPAECs were treated with 1 ng/ml IL-13 (Millipore Sigma, Catalog #H6291) for 8 hours.

Endothelial-monocyte cell adhesion assay. For endothelial to monocyte adhesion assay, CellTracker™ Green CMFDA Dye (Invitrogen™, Catalog #C2925) labelled monocytes were added on HPAECs stimulated by hypoxia-reoxygenation and IL-1β. After 90 minutes, floating cells were removed, and fluorescent imaging was performed with quantification of the adhered monocytes using Fiji image J.

Lactate measurements. HPAECs were treated with vehicle (DMSO) or syrosingopine (5 mM) for 24 hours. Then, cell media were collected, and lactate levels were measured by a Lactate assay kit (Sigma, Catalog #MAK064) following the kit's instruction and protocol.

### Statistical Analysis

The data were expressed as mean values±standard deviation of mean (SEM). Two-group comparison was performed by unpaired Student t-test with Welch's correction. Multigroup comparison was performed by one-way analysis of variance (ANOVA) with Sidak's multiple comparison test using GraphPad Prism 9 (GraphPad Software, La Jolla, CA). A value of P 0.05 was considered statistically significant, Significance is shown by *P<0.05, **P<0.01, ***P<0.001, ****P<0.0001. ScRNA-seq and snRNA-seq analyses were performed using the program “R” (http://cran.r-project.org/), “RStudio” (https://posit.co/download/rstudio-desktop/) (https://posit.co/products/open-source/rstudio/) and Seurat (v4.3.0).

### REFERENCES

1. Xu L, Guo J, Moledina D G, and Cantley L G. Immune-mediated tubule atrophy promotes acute kidney injury to chronic kidney disease transition. Nat Commun. 2022;13(1):4892.
2. Rudman-Melnick V, Adam M, Potter A, Chokshi S M, Ma Q, Drake K A, et al. Single-Cell Profiling of AKI in a Murine Model Reveals Novel Transcriptional Signatures, Profibrotic Phenotype, and Epithelial-to-Stromal Crosstalk. J Am Soc Nephrol. 2020; 31(12):2793-814.
3. Miao Z, Balzer M S, Ma Z, Liu H, Wu J, Shrestha R, et al. Single cell regulatory landscape of the mouse kidney highlights cellular differentiation programs and disease targets. Nat Commun. 2021;12(1):2277.
4. Klocke J, Kim S J, Skopnik C M, Hinze C, Boltengagen A, Metzke D, et al. Urinary single-cell sequencing captures kidney injury and repair processes in human acute kidney injury. Kidney Int. 2022; 102(6):1359-70.
5. Ransick A, Lindstrom N O, Liu J, Zhu Q, Guo J J, Alvarado G F, et al. Single-Cell Profiling Reveals Sex, Lineage, and Regional Diversity in the Mouse Kidney. Dev Cell. 2019; 51(3):399-413 e7.
6. McEvoy C M, Murphy J M, Zhang L, Clotet-Freixas S, Mathews J A, An J, et al. Single-cell profiling of healthy human kidney reveals features of sex-based transcriptional programs and tissue-specific immunity. Nat Commun. 2022;13(1):7634.
7. Liu W B, Huang G R, Liu B L, Hu H K, Geng J, Rui H L, et al. Single cell landscape of parietal epithelial cells in healthy and diseased states. Kidney Int. 2023; 104(1):108-23.
8. Muhl L, Genove G, Leptidis S, Liu J, He L, Mocci G, et al. Single-cell analysis uncovers fibroblast heterogeneity and criteria for fibroblast and mural cell identification and discrimination. Nat Commun. 2020;11(1):3953.
9. Kirita Y, Wu H, Uchimura K, Wilson P C, and Humphreys B D. Cell profiling of mouse acute kidney injury reveals conserved cellular responses to injury. Proc Natl Acad Sci USA. 2020; 117(27):15874-83.
10. Dumas S J, Meta E, Borri M, Goveia J, Rohlenova K, Conchinha N V, et al. Single-Cell RNA Sequencing Reveals Renal Endothelium Heterogeneity and Metabolic Adaptation to Water Deprivation. J Am Soc Nephrol. 2020; 31(1):118-38.
11. Tanigawa S, Tanaka E, Miike K, Ohmori T, Inoue D, Cai C L, et al. Generation of the organotypic kidney structure by integrating pluripotent stem cell-derived renal stroma. Nat Commun. 2022;13(1):611.
12. Conway B R, O'Sullivan E D, Cairns C, O'Sullivan J, Simpson D J, Salzano A, et al. Kidney Single-Cell Atlas Reveals Myeloid Heterogeneity in Progression and Regression of Kidney Disease. J Am Soc Nephrol. 2020; 31(12):2833-54.
13. Zimmerman K A, Bentley M R, Lever J M, Li Z, Crossman D K, Song C J, et al. Single-Cell RNA Sequencing Identifies Candidate Renal Resident Macrophage Gene Expression Signatures across Species. J Am Soc Nephrol. 2019; 30(5):767-81.
14. Wu H, Malone A F, Donnelly E L, Kirita Y, Uchimura K, Ramakrishnan S M, et al. Single-Cell Transcriptomics of a Human Kidney Allograft Biopsy Specimen Defines a Diverse Inflammatory Response. J Am Soc Nephrol. 2018; 29(8):2069-80.
15. Li H, Qu L, Yang Y, Zhang H, Li X, and Zhang X. Single-cell Transcriptomic Architecture Unraveling the Complexity of Tumor Heterogeneity in Distal Cholangiocarcinoma. Cell Mol Gastroenterol Hepatol. 2022; 13(5):1592-609 e9.
16. Bezman N A, Kim C C, Sun J C, Min-Oo G, Hendricks D W, Kamimura Y, et al. Molecular definition of the identity and activation of natural killer cells. Nat Immunol. 2012; 13(10):1000-9.
17. Morgan D, and Tergaonkar V. Unraveling B cell trajectories at single cell resolution. Trends Immunol. 2022; 43(3):210-29.
18. Liu Y, Hu J, Liu D, Zhou S, Liao J, Liao G, et al. Single-cell analysis reveals immune landscape in kidneys of patients with chronic transplant rejection. Theranostics. 2020; 10(19):8851-62.
19. Qu J, Yang F, Zhu T, Wang Y, Fang W, Ding Y, et al. A reference single-cell regulomic and transcriptomic map of cynomolgus monkeys. Nat Commun. 2022;13(1):4069.
20. Villani A C, Satija R, Reynolds G, Sarkizova S, Shekhar K, Fletcher J, et al. Single-cell RNA-seq reveals new types of human blood dendritic cells, monocytes, and progenitors. Science. 2017;356(6335).
21. Arazi A, Rao D A, Berthier C C, Davidson A, Liu Y, Hoover P J, et al. The immune cell landscape in kidneys of patients with lupus nephritis. Nat Immunol. 2019; 20(7):902-14.
22. Xie X, Shi Q, Wu P, Zhang X, Kambara H, Su J, et al. Single-cell transcriptome profiling reveals neutrophil heterogeneity in homeostasis and infection. Nat Immunol. 2020; 21(9):1119-33.

1. A method for treating acute kidney injury (AKI) in a subject in need thereof, the method comprising administering an agent that inhibits or reduces expression of MCT4.

2. The method of claim 1, wherein the agent is an MCT4 inhibitor.

3. The method of claim 2, wherein the agent is syrosingopine.

4. A method for treating fibrosis in a subject in need thereof, the method comprising administering an agent that inhibits or reduces expression of MCT4.

5. The method of claim 4, wherein the subject has elevated levels of a profibrotic protein.

6. The method of claim 5, wherein the profibrotic protein is expressed by Loxl2, Acta2, Vcam1, or Icam1.

7. The method of claim 4, wherein the agent is an MCT4 inhibitor.

8. The method of claim 7, wherein the agent is syrosingopine.

9. The method of claim 4, wherein the subject is in need of a treatment for AKI.

10. A method for treating inflammation in a subject in need thereof, the method comprising administering an agent that inhibits or reduces expression of MCT4.

11. The method of 10, wherein the subject has elevated levels of a proinflammatory protein.

12. The method of claim 11, wherein the proinflammatory protein is expressed by Tgfβ1.

13. The method of claim 10, wherein the agent is an MCT4 inhibitor.

14. The method of claim 13, wherein the agent is syrosingopine.

15. The method of claim 10, wherein the subject is in need of a treatment for AKI.

16. The method of claim 1 further comprising kidney dialysis.

17. The method of claim 1 further comprising intravenous administration of isotonic sodium chloride solution.

18. The method of claim 1 further comprising administration of at least one diuretic.

19. The method of claim 1 further comprising administration of at least one angiotensin-converting enzyme inhibitor.

20. The method of claim 1 further comprising administration of at least one angiotensin II receptor blocker.
